(19) United States
(12) Patent Application Publication
(10) Pub. No.: US 2009/0105261 A1
(43) Pub. Date: Apr. 23, 2009

(54) Novel substituted pyrazolo[1,5<I>A</I>]-1,3,5-Triazine derivatives and their analogues, pharmaceutical compositions containing same, use thereof as medicine and methods for preparing same

(75) Inventors: Philippe Bernard, Farges-En-Septaine (FR); Pierre Raboisson, Eckbolsheim (FR); Benoit Joseph, Villeurbanne (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: GREENPHARMA, Saint Beauzire (FR)

(21) Appl. No.: 12/315,201

(22) Filed: Dec. 1, 2008

Related U.S. Application Data
(63) Continuation of application No. 10/522,497, filed on Sep. 7, 2005, now abandoned.

(30) Foreign Application Priority Data
Jul. 26, 2002 (FR) 02/09519

(51) Int. Cl. A61K 31/53; C07D 251/72; A61P 25/00

(52) U.S. Cl. 514/246; 544/220

(57) ABSTRACT

The invention relates to novel derivatives capable of increasing the synthesis and/or the release of neurotrophic factors, and therefore able to be used as a human or veterinary medicinal product. The invention also relates to methods for preparing the derivatives and also to the intermediates required for their synthesis.

### CROSS-REFERENCE TO RELATED APPLICATIONS

This application is a continuation, of U.S. application Ser. No. 10/522,497, filed on Sep. 7, 2005, the disclosure of which is incorporated herein by reference.

### BACKGROUND OF THE INVENTION

The invention relates to novel derivatives capable in particular of increasing the synthesis and/or the release of neurotrophic factors, and therefore able to be used as a human or veterinary medicinal product, to methods for preparing them and also to the intermediates required for the synthesis.

Under physiological conditions, neurites (dendrites and axons) allow neurons to realize a large number of connections with neighboring neurons. These neurons, through the synapses, can transmit messages via messengers or neurotransmitters such as catecholamines, amino acids or peptides. When these connections between neurons become reduced, subsequent to cell death or to degeneration due to age or to diseases, disorders or traumas, the mental capacities of the individual can be seriously impaired.

Carbon monoxide, which is in particular produced by an enzyme, heme oxygenase, functions as a neurotransmitter and is capable of inducing, after diffusion into a cell, the production of a cellular second messenger: cyclic guanosine monophosphate (cGMP). This induction of cGMP is carried out by means of a carbon monoxide-dependent guanylate cyclase. Moreover, cGMP, just like cAMP, is degraded by a family of enzymes, phosphodiesterases (PDEs), that is divided up into at least 11 groups. PDE inhibitors, by slowing down the degradation of CGMP and of cAMP, increase or maintain the level of CGMP and of CAMP in cells and prolong their biological effects.

It is established that increasing intracellular cGMP levels results in a modification of many cellular activities, and in particular of the synthesis and release of several endogenous neurotrophic factors (neurotrophin and pleiotrophin) and also of other neuronal factors which can induce, promote or modify a large variety of cell functions, in particular cell growth and cell communication.

Neurotrophic factors are molecules which exert a very large variety of biological effects and stimulate the development and differentiation of neurons and the maintenance of cell integrity, and which are required for neuron survival and neuron development. More particularly, neurotrophic factors make it possible to prevent neuronal death and to stimulate neurite growth and also to decrease membrane potentials, making the neuron more receptive to cell signals. Growth factors can also change the long-term potentiation of neurons, inducing an increase in neuronal plasticity and making it possible to increase cognitive and mental faculties.

In certain states or certain central or peripheral diseases, neuronal functions are impaired. Among these states or diseases most commonly resulting from excessive neuronal death, mention may in particular be made, without implied limitation, of: aging, Alzheimer's disease, Parkinson's disease, amyotrophic lateral sclerosis, multiple scleroses, Huntington's disease, cerebral strokes, peripheral neuropathies, retinopathies (in particular pigmentary retinitis), prion diseases (in particular spongiform encephalopathies of the Creutzfeldt-Jakob disease type), traumas (accidents to the vertebral column, compression of the optic nerve subsequent to a glaucoma, etc.) or else neuronal disorders caused by the action of chemical products, and also the disorders associated with these states or diseases, which may be disorders that are secondary to the primary pathology. In many cited cases, it is most commonly the progressive death of motoneurons which will be the cause of the disorders observed, and conventional treatments make use of the administration of anti-inflammatory agents in order to prevent the occurrence of secondary disorders.

One of the means of preventing such impairments and/or of re-establishing a neuronal function that has been damaged is to regenerate neurites between the various nerve cells, for example by increasing the local concentrations of one or more growth factors. Treatments that make use of small molecules capable of increasing the synthesis and/or the secretion of growth factors and that preferentially act by oral or injectable administration will be preferred to those using natural growth factors, which are large molecules that are inactive orally and are incapable of penetrating the central nervous system. These small molecules, by inducing the secretion and/or the synthesis of growth factors, are also capable of changing the long-term potentiation of neurons, inducing, in particular in the hippocampus, an increase in neuronal plasticity, the consequence of which will be to increase the cognitive and mental faculties.

Furthermore, inhibitors of phosphodiesterases type 2 and 4 (PDE2 and PDE4) are capable, by increasing the intracellular concentration of cAMP, of exerting a cytoprotective effect and of increasing in particular the survival of dopaminergic neurons (Pérez-Torres, S. et al., J. Chem. Neuroanatomy, 2000, 20, 349-374). It has also been reported that cAMP is involved in the transduction of many neurotransmitters and hormones and can thus in particular modulate the effect of growth factors. An inhibitor of PDE4 or of PDE2, by slowing down cAMP degradation, can consequently produce a neurological and/or neuroprotective effect. It is, moreover, known that PDE4 inhibitors represent potential treatments for many central or peripheral diseases, in particular autoimmune and inflammatory diseases. The field of application of PDE4 inhibitors covers in particular the treatment and prevention of inflammation and of a lack of bronchial relaxation, and more particularly of asthma and of chronic obstructive bronchopathies, but also of other conditions such as rhinitis, acute respiratory distress syndrome, allergies, dermatitis, psoriasis, rheumatoid arthritis, multiple scleroses (in particular multiple sclerosis), dyskinesias, glomerulonephritis, osteoarthritis, cancer, septic shock, AIDS, Crohn's disease, osteoporosis, rheumatoid arthritis or obesity. PDE4Is also have central effects that are particularly advantageous for the treatment of depression, of anxiety, of schizophrenia, of bipolar disorder, of attention deficits, of fibromyalgia, of Parkinson's disease and Alzheimer's disease, of amyotrophic sclerosis, of multiple scleroses, of Lewy body dementias and of other psychiatric disorders.

International application WO 99/67247 describes pyrazolotriazines, CRF antagonists, corresponding to the general formula:

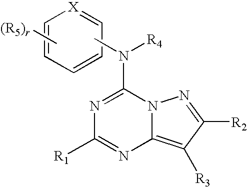

in which the hexocyclic nitrogen atom in the 4-position necessarily carries a phenyl or pyridyl aromatic group. International application WO 99/67247 does not therefore disclose pyrazolo[1,5-a]-1,3,5-triazines that are identical to those claimed in the present invention.

International application WO 99/38868 describes pyrazolotriazines, CRF antagonists, corresponding to the general formula:

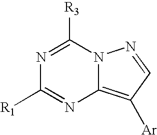

in which the substituent in the 8-position is necessarily a pyridyl or phenyl aromatic group. International application WO 99/38868 does not therefore disclose pyrazolo[1,5-a]-1,3,5-triazines that are identical to those claimed in the present invention.

Other pyrazolotriazines that are CRF antagonists are described in international application WO 98/03510 and correspond to the general formula:

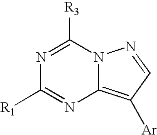

in which the group in the 8-position is necessarily aromatic and is chosen from phenyl, naphthyl, pyridyl, pyrimidyl, triazinyl, furanyl, thienyl, benzothienyl, benzofuranyl, 2,3-dihydrobenzofuranyl, 2,3-dihydrobenzothienyl, indanyl, 1,2-benzopyranyl, 3,4-dihydro-1,2-benzopyranyl and tetralinyl. International application WO 98/03510 does not therefore disclose pyrazolo[1,5-a]-1,3,5-triazines that are identical to those claimed in the present invention.

U.S. Pat. No. 4,183,930 describes pyrazolotriazines corresponding to the general formula:

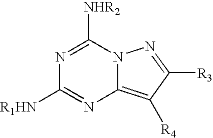

in which the substituent in the 2-position is necessarily a group NHR₁, R₁ being a hydrogen atom or a (C₁-C₄)alkyl radical, where R₄ is a hydrogen atom or a (C₁-C₄)alkyl group. These compounds have in particular bronchodilatory, anti-allergic and neurological properties, but also have hypotensive properties which may be prejudicial. In addition, U.S. Pat. No. 4,183,930 does not disclose pyrazolo[1,5-a]-1,3,5-triazines that are identical to those claimed in the present invention.

Application EP 515 107 describes compounds corresponding to the general formula:

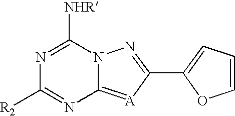

in which the substituent in the 7-position is necessarily a 2-furyl group and A represents either a nitrogen atom or a group CT where T is a hydrogen atom or a (C₁-C₄)alkyl group. These compounds antagonize the effect of adenosine. Application EP 515 107 does not therefore disclose pyrazolo[1,5-a]-1,3,5-triazines that are identical to those claimed in the present invention.

International application WO 00/59907 describes CRF antagonists corresponding to the general formula:

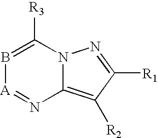

in which the compound is a pyrazolo[1,5-a]-1,3,5-triazine when A represents CR5 and B represents N. The compounds described are, however, limited to pyrazolo[1,5-a]-1,3,5-triazines in which R3 is necessarily an aryl or heteroaryl group. International application WO 00/59907 does not therefore disclose pyrazolo[1,5-a]-1,3,5-triazines that are identical to those claimed in the present invention.

Application FR 2 818 278 and international application WO 02/50079 describe pyrazolo[1,5-a]-1,3,5-triazines, that inhibit cyclin-dependent kinases (CDKs), corresponding to the general formula:

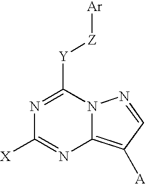

in which Y represents NH or O; Z represents a bond or an alkyl or thioalkyl group and Ar represents an optionally substituted carbocyclic aryl radical. Applications FR 2 818 278 and WO 02/50079 do not therefore disclose pyrazolo[1,5-a]-1,3,5-triazines that are identical to those claimed in the present invention.

Application EP 0269859 describes pyrazolo[1,5-a]-1,3,5-triazines, that are xanthine oxidase inhibitors, corresponding to the general formula:

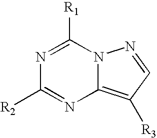

in which R1 is necessarily a hydroxyl or a C₁-C₆ alkanoyloxy group and R2 is necessarily a hydrogen, and R3 is an unsaturated heterocycle. Application EP 0269859 does not therefore disclose pyrazolo[1,5-a]-1,3,5-triazines that are identical to those claimed in the present invention.

U.S. Pat. No. 3,910,907 describes pyrazolo[1,5-a]-1,3,5-triazines, that are cAMP phosphodiesterase inhibitors, corresponding to the general formula:

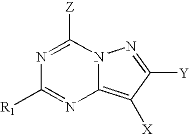

in which R1 is necessarily a group CH3, C2H5 or C6H5; X is chosen from H, C6H5, (m)CH3C6H4, CN, COOEt, Cl, I or Br; Y represents H, C6H5, (O)CH3C6H4 or (p)CH3OC6H4, and Z represents H, OH, CH3, C2H5, C6H5, n-C3H7, iso-C3H7, SH, SCH3, NH(n-C4H9) or N(C2H5)2. These phosphodiesterase inhibitors are therefore different from those reported in the present invention.

U.S. Pat. No. 3,995,039 describes other pyrazolo[1,5-a]-1,3,5-triazines, that are cAMP phosphodiesterase inhibitors, corresponding to the general formula:

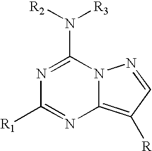

in which R is necessarily a heterocycle directly attached at the 8-position of the pyrazolotriazine ring, R1 and R2 represent alkyl or hydrogen, and R3 is chosen from a hydrogen atom, or an alkyl, alkanoyl, carbamoyl or N-alkylcarbamoyl group. These phosphodiesterase inhibitors are therefore different from those reported in the present invention, and also have, along with a bronchodilatory activity, hypotensive properties which may be prejudicial to their use in human therapeutics. Moreover, no selectivity with respect to type 2 and type 4 phosphodiesterases was reported for these compounds.

Other pyrazolo[1,5-a]-1,3,5-triazines that are phosphodiesterase inhibitors are described in the article according to J. Med. Chem. 1982, 25, 243-249, and correspond to the general formula:

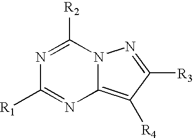

in which R4 represents Br or H, R1 is chosen from H, CH3 or SCH3, R4 is C6H5 or H, and R2 represents SCH3, NH(n-Pr), NH(n-Bu), N(Et)₂, piperidyl, OH, SH, O(i-Pr), CH₃, SEt, OCH3 or O(n-Pr). These PDE inhibitors are therefore also different from those disclosed in the present invention. Moreover, no selectivity with respect to type 2 and type 4 phosphodiesterases was reported for these compounds.

Other PDE4 inhibitors with a pyrazolo[1,5-a]-1,3,5-triazine structure are described in a thesis from the University of Strasbourg I: Pierre Raboisson, “Développement d'inhibiteurs de Phosphodiestérase 4 and conception d'antagonistes purinergiques P2Y1 à partir de dérivés de l'adénine et de leurs analogues structuraux” [Development of phosphodiesterase 4 inhibitors and design of P2Y1 purinergic antagonists based on derivatives of adenine and their structural analogues], Nov. 27, 2000. Although powerful inhibitors have been developed with respect to bovine PDE4, no datum concerning: (i) the activity of these molecules on human PDE4, (ii) the absence of emetic effect of these compounds on an animal model, or (iii) proof of effectiveness on a model of asthma or other model of inflammatory or autoimmune disease, or (iv) the selectivity of these compounds with respect to PDE6, was reported. In addition, the compounds described in this thesis showed a limited effect on TNFα secretion (only four compounds tested with an IC50 which is greater than 100 nM) and no parallel was observed between inhibition of PDE4 and the decrease in TNFα secretion, implying that these compounds may act on a biological target other than PDE4. Moreover, no neurotrophic effect was observed with these compounds. In addition, these compounds are different from those disclosed in the present invention.

### BRIEF DESCRIPTION OF THE FIGURES

FIG. 1 illustrates the effect of a molecule of Ia5 on neurons and its effect on neurite development

### DETAILED DESCRIPTION

The applicant has now demonstrated that the compounds according to the invention are capable of increasing the synthesis and/or the release of one or more endogenous neurotrophic factors. Some compounds according to the invention also have PDE2- or PDE4-inhibiting properties.

Consequently, a subject of the invention is compounds corresponding to general formula (I)

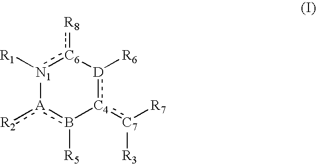

in which:
A represents C or N,
B and D, which may be identical or different, are chosen from N or C, with the proviso that A and B do not simultaneously represent a nitrogen atom,
R₁ represents
either a hydrogen atom,
or a (C₁-C₁₂)alkyl, (C₃-C₆)cycloalkyl, (C₆-C₁₈)aryl, (C₆-C₁₈)aryl(C₁-C₄)alkyl, (C₁-C₁₂)alkyl(C₆-C₁₈)aryl, (C₂-C₈)alkenyl, (C₂-C₈)alkynyl, (C₁-C₈)alkoxy or hydroxyl group,
or an aromatic or nonaromatic (C₅-C₁₈)heterocycle containing from 1 to 3 hetero atoms and being attached directly to the nitrogen atom in the 1-position by means of a single bond or by means of a (C₁-C₆)alkyl, (C₂-C₆)alkenyl or (C₂-C₆)alkynyl group,
or a group NR′R″ or NHCOR′R″, R′ and R″, independently of one another, being chosen from a hydrogen atom, (C₁-C₆)alkyl, (C₃-C₆)cycloalkyl and (C₆-C₁₂)aryl groups, and aromatic or nonaromatic (C₅-C₁₂)heterocycles containing from 1 to 3 hetero atoms;
   R₂ and R₃, which may be identical or different, each represent
either a hydrogen atom,
or a halogen atom,
or a group: (C₁-C₆)alkoxy, (C₁-C₁₀)alkyl, (C₁-C₆)alkylCOOH, (C₁-C₆)alkylCOONa, perfluoro(C₁-C₆)alkyl, (C₃-C₆)cycloalkyl, acyl, (C₂-C₆)alkenyl, (C₂-C₆)alkynyl, (C₆-C₁₈)aryl, (C₆-C₁₈)arylCOOH, (C₆-C₁₈)arylCOONa, (C₆-C₁₈)aryl(C₁-C₄)alkyl, (C₁-C₆)alkyl(C₆-C₁₈)aryl, (C₅-C₁₈)heteroaryl, (C₁-C₆)alkyl(C₅-C₁₈)heteroaryl, (C₂-C₆)alkenyl(C₅-C₁₈)heteroaryl, (C₂-C₆)alkynyl(C₅-C₁₈)heteroaryl, CH(OH)(C₆-C₁₈)aryl, CO(C₆-C₁₈)aryl, (CH₂)ₙCONH— (CH₂)ₘ—(C₆-C₁₈)aryl, (CH₂)ₙSO₂NH—(CH₂)ₘ—(C₆-C₁₈)aryl or (CH₂)ₙCONH—CH(COOH)—(CH₂)ₚ—(C₆-C₁₈)aryl with n=1 to 4, m=0 to 3 and p=0 to 2, in which one or more groups —CH₂— can be optionally replaced with —O—, —S—, —S(O)—, —S(O)₂— or —NH—, and can be optionally substituted with one or more radicals chosen from the following radicals: (C₁-C₆)alkyl, hydroxyl, oxo, (C₆-C₁₈)aryl(C₁-C₈)alkyl, (C₆-C₁₈)aryl, halogen, cyano, phosphate, alkylphosphate, nitro, alkoxy, (C₅-C₁₈)heteroaryl, (C₅-C₁₈)heteroaryl(C₁-C₆)alkyl, COOH, CONRₓR_{y}, NRₓCONHR_{y}, ORₓ, SRₓ, SORₓ, SO₂Rₓ, CORₓ, COORₓ, NRₓSO₂R_{y} or NRₓR_{y} in which (i) Rₓ and R_{y}, independently of one another, are chosen from a hydrogen atom and the following groups: (C₁-C₆)-alkyl, (C₃-C₆)cycloalkyl, (C₆-C₁₈)aryl, (C₆-C₁₈)aryl(C₁-C₄)alkyl, (C₁-C₁₂)alkyl(C₆-C₁₈)aryl, (C₃-C₆)cycloalkyl(C₆-C₁₂)aryl, (C₁-C₆)alkoxy(C₁-C₆)alkyl, (C₅-C₁₂)heteroaryl containing 1 to 3 hetero atoms, OR′, NR′R″ and NHCOR′R″, R′ and R″, independently of one another, being chosen from a hydrogen atom, (C₁-C₆)alkyl, (C₃-C₆)cycloalkyl and (C₆-C₁₂)aryl groups, and aromatic or nonaromatic (C₅-C₁₂)heterocycles containing 1 to 3 hetero atoms, or (ii) Rₓ and R_{y} together form a linear or branched hydro-carbon-based chain having from 2 to 6 carbon atoms, optionally containing one or more double bonds and/or optionally interrupted with an oxygen, sulfur or nitrogen atom,
or a nitro, cyano, ORₓ, SRₓ, SORₓ, SO₂Rₓ, CORₓ, CONRₓR_{y}, COORₓ, NRₓCOR_{y}, NRₓSO₂R_{y} or NRₓR_{y} group in which Rₓ and R_{y} are as defined above,
it being understood that, in the definition of the groups R₂ and R₃, the “aryl” groups can be replaced with aromatic or nonaromatic C₄-C₁₀ “heterocycles” containing from 1 to 3 hetero atoms;
   R₅ represents
either a hydrogen atom,
or a group: (C₁-C₆)alkyl, (C₃-C₆)cycloalkyl(C₆-C₁₂)aryl, or (C₅-C₁₂)heteroaryl containing 1 to 3 hetero atoms;
   R6 and R7 form, together with the atoms which carry them, a 5- or 6-membered ring which may contain another hetero atom chosen from the group consisting of N, O and S, and in which

if the bond between N₁ and C6 is a single bond, then the bond between C6 and R8 is a double bond and R₈═X, where X represents either an oxygen or sulfur atom, or a group NRₓ in which Rₓ is as defined above,

if the bond between N1 and C6 is a double bond, then the bond between C6 and R8 is a single bond and R8=Y where Y represents either a halogen atom, or a (C1-C6)alkyl, (C2-C6)alkenyl, (C2-C6)alkynyl, (C1-C6)alkoxy, (C3-C6)cycloalkyl, ORx, SRx, SORx, SO2Rx, NRxCORy, NRxSO2Ry or NRxRy group in which Rx and Ry are as defined above and R1 is not present,

if the bond between A and B is a single bond, then the bond between A and R2 is a double bond and R2=X where X is as defined above, and

if the bond between A and B is a double bond, then the bond between A and R2 is a single bond, R2 is as defined above and R5 is not present,

if the bond between C4 and D is a single bond, then the bond between C4 and C7 is a double bond,

if the bond between C4 and D is a double bond, then the bond between C4 and C7 is a single bond, and D is a carbon atom, or else D is a nitrogen atom and R6 is not present,

their tautomeric forms, their isomers, diastereoisomers and enantiomers, their prodrugs, their bioprecursors and their pharmaceutically acceptable base or acid addition salts, with the proviso that, when the compounds correspond to formula (Ia)

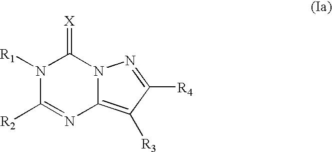

### or (Ib)

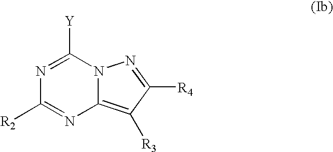

then

when Y, in formula (Ib), represents ORx, then Rx is necessarily different from aryl and aralkyl;

when simultaneously, in formula (Ib), Y represents NRxRy and Rx represents H, then Ry is necessarily different from aryl and aralkyl;

when Y, in formula (Ib), represents a group NRxRy in which at least one of the groups Rx or Ry is chosen from optionally substituted phenyl or pyridyl groups, then R3 is different from a (C1-C10)alkyl, (C2-C10)alkenyl, (C2-C10)alkynyl, (C3-C8)cycloalkyl and (C3-C6)cycloalkyl(C1-C4)alkyl group, it being possible for the latter to be optionally substituted;

when R3, in formula (Ib), represents an optionally substituted phenyl or pyridyl group, then Y is different from: NHCH(CH2CH2OMe)(CH2OMe), NHCH(Et)2,2-ethylpiperid-1-yl, cyclobutylamino, N(Me)CH2CH═CH2, N(Et)CH2CH═CH2, N(Me)CH2cPr, N(Et)CH2cPr, N(Pr)CH2cPr, N(Me)Pr, N(Me)Et, N(Me)Bu, N(Me)propargyl, N(Et)propargyl, NHCH(CH3)CH(CH3)CH3, N(CH2CH2OMe)CH2CH═CH2, N(CH2CH2OMe)Me, N(CH2CH2OMe)Et, N(CH2CH2OMe)Pr, N(CH2CH2OMe)CH2cPr, NHCH(CH3)CH2CH3, NHCH(cPr)2, N(CH2CH2OMe)2, N(Et)2 and cyclobutylamino;

when R3, in formula (Ib), represents a phenyl, naphthyl, pyridyl, pyrimidyl, triazinyl, furanyl, thienyl, benzothienyl, benzofuranyl, 2,3-dihydrobenzofuranyl, 2,3-dihydrobenzothienyl, indanyl, 1,2-benzopyranyl, 3,4-dihydro-1,2-benzopyranyl or tetralinyl group, then R1 in formula (Ia) is different from H;

when simultaneously, in formula (Ib), R3 represents a heterocycle directly attached at the 8-position of the pyrazolotriazine ring, R2 represents alkyl or hydrogen, and Y represents a group NRxRy, Rx being chosen from a hydrogen atom or an alkyl group, then Ry is different from H or from an alkyl, alkanoyl, carbamoyl or N-alkylcarbamoyl group;

when NRxRy, in formula (Ib), represents an NH2 group or a group NH(C1-C4)alkyl, then R4 is different from a hydrogen atom or a C1-C4 alkyl group;

when simultaneously, in formula (Ib), Y represents NHCH3, R2 represents CH3 and R4 represents a hydrogen atom, then R3 is different from benzyl, phenyl, naphthyl, (2-naphthyl)methyl, pentyl, benzoyl, propyne, penten-1-yl, 2-furyl, 2-thienyl, 2-chlorophenyl, 3-acetylphenyl, 3-nitrophenyl, 3-trifluoromethylphenyl, 2-benzo[b]furyl, 2-benzo[b]thienyl, 2-chlorobenzoyl, 2-methylaminobenzoyl, 4-methoxybenzoyl, 3-trifluoromethylbenzoyl, furfuryl, (3-furyl)methyl, (2-thienyl)methyl, 2-hydroxypropyl, iodo, nitro, acetylamino, benzoylamino and diethylaminocarbonyl;

when simultaneously, in formula (Ib), Y represents NHCH3, R4 represents H and R3 represents benzoyl or iodo, then R2 is different from methyl, ethyl, n-propyl, n-butyl, thiomethyl, methoxymethyl, phenyl and 2-furyl;

when simultaneously, in formula (Ib), Y represents NHCH3, R4 represents H and R3 represents benzyl or 2-methoxybenzyl, then R2 is different from methyl, n-propyl and trifluoromethyl;

when simultaneously, in formula (Ib), Y represents a methylamino, benzylamino, pyrrolidinyl, dimethylamino or 1-piperazinyl group and R2 represents methyl or n-propyl, then R3 is different from iodo and benzoyl;

when R4, in formula (Ib), is a 2-furyl group, then R3 is different from a hydrogen atom or from a (C₁-C₄)alkyl group;

when simultaneously, in formulae (Ia) and (Ib), R1 is a hydrogen atom with R2 chosen from CH3, C2H5 or C6H5, R3 is chosen from H, C6H5, (m)CH3C6H4, CN, COOEt, Cl, I or Br, and R4 represents H, C6H5, (O)CH3C6H4 or (p)CH3OC6H4, then Y is different from H, OH, CH3, C2H5, C6H5, n-C3H7, iso-C3H7, SH, SCH3, NH(n-C4H9) or N(C2H5)2 and X is different from O;

when simultaneously, in formula (Ib), R1 represents H, R3 represents Br or H, and R2 is chosen from H, CH3 or SCH3 with R4 being C6H5 or H, then Y is different from SCH3, NH(n-Pr), NH(n-Bu), N(Et)2, piperidyl, OH, SH, O(i-Pr), CH3, SEt, OCH3 and O(n-Pr);

when simultaneously, in formula (Ib), R₂ represents CF₃, CH₃OCH₂—, Ph, Et, n-Pr or CH₃, Y represents NHCH₃, N(CH₃)₂ or N(CH₃)Ph, and R₄═H or CH₃, then R₃ is different from β-D-glycero-pentofuran-3′-ulos-1′-yl, 2′-deoxy-β-D-ribofuranosyl, 2′-deoxy-β-D-xylofuranosyl, 2′-deoxy-β-D-ribofuranosyl-3′,5′-bis(dibenzyl phosphate), cyclic benzyl 2′-deoxy-β-D-xylofuranosyl-3′,5′-phosphate, 2′-deoxy-β-D-ribofuranosyl-3′,5′-bisphosphate and cyclic 2′-deoxy-β-D-xylofuranosyl-3′,5′-phosphate.

Advantageously, the compounds correspond to formula (I) in which A is a carbon atom, and B and D are nitrogen atoms, the 6-membered heterocycle thus formed being a 1,3,5-triazine.

If A and D represent carbon atoms and B is a nitrogen atom, then the 6-membered heterocycle is a pyrimidine, for example a derivative of uracil or of cytosine.

It is understood that, in formula (I), the C4 carbon atom can be advantageously replaced with a nitrogen atom in the following case: when the compounds correspond to formula (I) in which A is a carbon atom and B is a nitrogen atom.

Thus, the 6-membered heterocycle thus formed is a 1,2,4-triazine. These compounds are particularly advantageous when the 5-membered fused ring is an imidazole. In this case, the bicycle of formula (I) will be an imidazotriazine.

Very advantageously, the compounds according to the invention correspond more particularly to formula (Ia)

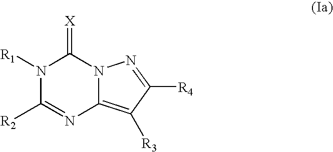

or to formula (Ib)

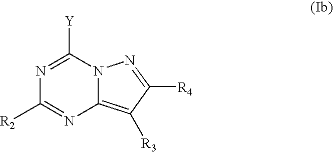

in which:
R₁, R₂, R₃, X and Y are as defined above, and
R₄ represents:
either a hydrogen atom, a (C₁-C₁₂)alkyl, (C₃-C₆)cycloalkyl, (C₆-C₁₈)aryl, (C₆-C₁₈)aryl(C₁-C₄)alkyl or (C₁-C₁₂)alkyl(C₆-C₁₈)aryl group, or an aromatic or nonaromatic (C₅-C₁₈)heterocycle containing 1 to 3 hetero atoms, in which one or more groups —CH₂— can be optionally replaced with —O—, —S—, —S(O)—, —S(O)₂— or —NH—, and can be optionally substituted with one or more radicals chosen from (C₁-C₆)alkyl, hydroxyl, oxo, halogen, cyano, nitro and alkoxy radicals,

or a group NR′R″ or NHCOR′R″, R′ and R″, independently of one another, being chosen from a hydrogen atom, a (C1-C6)alkyl, (C3-C6)cycloalkyl or (C6-C12)aryl group, and an aromatic or nonaromatic (C5-C12)heterocycle containing from 1 to 3 hetero atoms, it being possible for said formulae (Ia) and (Ib) to be, with respect to one another, tautomeric forms according to the definition of R1, of X and of Y, with the proviso that:

when Y, in formula (Ib), represents ORx, then Rx is necessarily different from aryl and aralkyl;

when simultaneously, in formula (Ib), Y represents NRxRy and Rx represents H, then Ry is necessarily different from aryl and aralkyl;

when Y, in formula (Ib), represents a group NRₓR_{y} in which at least one of the groups Rₓ or R_{y} is chosen from optionally substituted phenyl or pyridyl groups, then R₃ is different from a (C₁-C₁₀)alkyl, (C₂-C₁₀)alkenyl, (C₂-C₁₀)alkynyl, (C₃-C₈)cycloalkyl and (C₃-C₆)cycloalkyl(C₁-C₄)alkyl group, it being possible for the latter to be optionally substituted;

when R3, in formula (Ib), represents an optionally substituted phenyl or pyridyl group, then Y is different from: NHCH(CH2CH2OMe)(CH2OMe), NHCH(Et)2,2-ethylpiperid-1-yl, cyclobutylamino, N(Me)CH2CH═CH2, N(Et)CH2CH═CH2, N(Me)CH2cPr, N(Et)CH2cPr, N(Pr)CH2cPr, N(Me)Pr, N(Me)Et, N(Me)Bu, N(Me)propargyl, N(Et)propargyl, NHCH(CH3)CH(CH3)CH3, N(CH2CH2OMe)CH2CH═CH2, N(CH2CH2OMe)Me, N(CH2CH2OMe)Et, N(CH2CH2OMe)Pr, N(CH2CH2OMe)CH2cPr, NHCH(CH3)CH2CH3, NHCH(cPr)2, N(CH2CH2OMe)2, N(Et)2 and cyclobutylamino;

when R3, in formula (Ib), represents a phenyl, naphthyl, pyridyl, pyrimidyl, triazinyl, furanyl, thienyl, benzothienyl, benzofuranyl, 2,3-dihydrobenzofuranyl, 2,3-dihydrobenzothienyl, indanyl, 1,2-benzopyranyl, 3,4-dihydro-1,2-benzopyranyl or tetralinyl group, then R1 in formula (Ia) is different from H;

when simultaneously, in formula (Ib), R3 represents a heterocycle directly attached at the 8-position of the pyrazolotriazine ring, R2 represents alkyl or hydrogen, and Y represents a group NRxRy, Rx being chosen from a hydrogen atom or an alkyl group, then Ry is different from H or from an alkyl, alkanoyl, carbamoyl or N-alkylcarbamoyl group;

when NRxRy, in formula (Ib), represents an NH2 group or a group NH(C₁-C₄)alkyl, then R4 is different from a hydrogen atom or a C₁-C₄ alkyl group;

when simultaneously, in formula (Ib), Y represents NHCH₃, R2 represents CH₃ and R4 represents a hydrogen atom, then R3 is different from benzyl, phenyl, naphthyl, (2-naphthyl)methyl, pentyl, benzoyl, propyne, penten-1-yl, 2-furyl, 2-thienyl, 2-chlorophenyl, 3-acetylphenyl, 3-nitrophenyl, 3-trifluoromethylphenyl, 2-benzo[b]furyl, 2-benzo[b]thienyl, 2-chlorobenzoyl, 2-methylaminobenzoyl, 4-methoxybenzoyl, 3-trifluoromethylbenzoyl, furfuryl, (3-furyl)methyl, (2-thienyl)methyl, 2-hydroxypropyl, iodo, nitro, acetylamino, benzoylamino and diethylaminocarbonyl;

when simultaneously, in formula (Ib), Y represents NHCH3, R4 represents H and R3 represents benzoyl or iodo, then R2 is different from methyl, ethyl, n-propyl, n-butyl, thiomethyl, methoxymethyl, phenyl and 2-furyl;

when simultaneously, in formula (Ib), Y represents NHCH₃, R4 represents H and R3 represents benzyl or 2-methoxybenzyl, then R2 is different from methyl, n-propyl and trifluoromethyl;

when simultaneously, in formula (Ib), Y represents a methylamino, benzylamino, pyrrolidinyl, dimethylamino or 1-piperazinyl group and R2 represents methyl or n-propyl, then R3 is different from iodo and benzoyl;

when R4, in formula (Ib), is a 2-furyl group, then R3 is different from a hydrogen atom or from a (C1-C4)alkyl group;

when simultaneously, in formulae (Ia) and (Ib), R1 is a hydrogen atom with R2 chosen from CH3, C2H5 or C6H5, R3 is chosen from H, C6H5, (m)CH3C6H4, CN, COOEt, Cl, I or Br, and R4 represents H, C6H5, (O)CH3C6H4 or (p)CH3OC6H4, then Y is different from H, OH, CH3, C2H5, C6H5, n-C3H7, iso-C3H7, SH, SCH3, NH(n-C4H9) or N(C2H5)2 and X is different from O;

when simultaneously, in formula (Ib), R1 represents H, R3 represents Br or H, and R2 is chosen from H, CH3 or SCH3 with R4 being C6H5 or H, then Y is different from SCH3, NH(n-Pr), NH(n-Bu), N(Et)2, piperidyl, OH, SH, O(i-Pr), CH3, SEt, OCH3 and O(n-Pr);

when simultaneously, in formula (Ib), R2 represents CF3, CH3OCH2-, Ph, Et, n-Pr or CH3, Y represents NHCH3, N(CH3)2 or N(CH3)Ph, and R4=H or CH3, then R3 is different from β-D-glycero-pentofuran-3′-ulos-1′-yl, 2′-deoxy-β-D-ribofuranosyl, 2′-deoxy-β-D-xylofuranosyl, 2′-deoxy-β-D-ribofuranosyl-3′,5′-bis(dibenzyl phosphate), cyclic benzyl 2′-deoxy-β-D-xylofuranosyl-3′,5′-phosphate, 2′-deoxy-β-D-ribofuranosyl-3′,5′-bisphosphate and cyclic 2′-deoxy-β-D-xylofuranosyl-3′,5′-phosphate.

In a particularly advantageous embodiment of the invention,

R₁ represents either a hydrogen atom or a (C₁-C₁₂)alkyl group,

R2 represents either a hydrogen or sulfur atom, or a (C1-C6)alkyl group, or a trifluoro(C1-C6)alkyl group, or an amino group, or a group SRx where Rx is as defined above,

R3 represents either a hydrogen atom, or a halogen atom, or a nitro, (C1-C6)alkyl, trifluoro(C1-C6)alkyl, acyl, (C2-C6)alkenyl, (C2-C6)alkynyl, (C6-C18)aryl, (CH2)nCONH—

(CH2)maryl, (CH2)nSO2NH—(CH2)maryl or (CH2)nCONH—CH(COOH)—(CH2)paryl group with n=1 to 4, m=0 to 3 and p=0 to 2, or a group NR′R″ or NHCOR′R″, R′ and R″, independently of one another, being chosen from a hydrogen atom, (C1-C6)alkyl, (C3-C6)cycloalkyl and (C6-C12)aryl groups, and aromatic or nonaromatic (C5-C12)heterocycles containing 1 to 3 hetero atoms,

R4 represents a hydrogen atom,

X represents an oxygen or sulfur atom, and

Y represents either a halogen atom, or a (C₁-C₆)alkyl, (C₂-C₆)alkynyl, phenyl, ORₓ, SRₓ or NRₓR_{y} group in which Rₓ and R_{y} are as defined above.

Even more advantageously,

R₁ represents a hydrogen atom or a methyl group,

R₂ represents a hydrogen or sulfur atom, or a methyl, propyl, trifluoromethyl, amino or thiomethyl group,

R₃ represents an iodine atom, or an amino, nitro, acylamino, benzyl, 2-methoxybenzyl, furfuryl, 3-furylmethyl, 2-thienylmethyl, 3-thienylmethyl, 2-pyridylmethyl, 2-chlorobenzoyl —CH₂CH₂COOH, CH₂CH₂COONa, C₆H₄COOH, C₆H₄COONa, C₆H₄COOC₂H₅, ethyl benzoate, sodium benzoate, CH₂═CHCOOC₂H₅, propyn-1-yl, (CH₂)₂CONH—C₆H₄COONa, (CH₂) CONH—(CH₂)₂-indole, (CH₂)₂CONH—CH(COOH)(CH₂)indole, (CH₂)CONH— (CH₂)₂C₆H₄OH or (CH₂)₂CONH—CH₂C₆H₄OH group,

X represents an oxygen atom, and

Y represents an OH, SH, N-methyl-N-phenylamino (NPhCH₃), N-methyl-N-(4-acylaminophenyl)amino or triazole group.

In a particularly advantageous embodiment of the invention, a subject of said invention is the compounds corresponding to formulae (Ic₁) and (Ic₂)

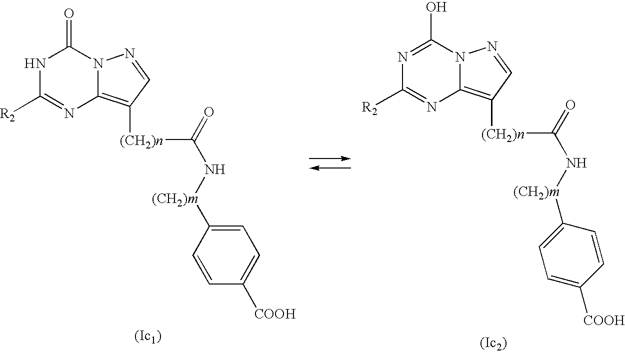

in which n=1 to 4, and m=0 to 2,

and also their prodrugs, their bioprecursors and their pharmaceutically acceptable base or acid addition salts.

Even more advantageously, in the compounds of formulae (Ic1) and (Ic2), R2 represents a hydrogen atom, n=2 and m=0.

These compounds were found to be powerful stimulators of neuronal growth. The compound sodium 4-[[1-(oxo)-3-(4-oxopyrazolo[1,5-a]-1,3,5-triazin-8-yl)-propyl]amino]benzoate (Ia5) corresponding to formulae Ic1 and Ic2 in which R2=H, n=2 and m=0 is particularly preferred.

Other compounds that are particularly advantageous for the purpose of the invention are those corresponding to the general structure (Ib) in which Y represents a methylamino or cyclopropylamino group, R2 represents an iodine or sulfur atom, or a methyl, propyl, cyclopropyl, perfluoroethyl, perfluoropropyl, trifluoromethyl, allyl, trifluoromethylvinyl, vinyl, 1-propynyl or ethynyl group, and R4 represents a hydrogen or fluorine atom. These compounds are very powerful and very selective inhibitors of PDE4. In this case, and advantageously, R3 will, for example, be chosen from an iodine atom, and a benzyl, 2-methoxybenzyl, 2-fluorobenzyl, 2-bromobenzoyl, furfuryl, 2-furylcarbonyl, 3-furylmethyl, 2-thienylmethyl, 3-thienylmethyl, 2-pyridylmethyl, 2-chlorobenzoyl, cyclopentyl or cyclohexyl group.

Other compounds that are advantageous for the purpose of the invention are those corresponding to the structures (Ia) and (Ib) in which X represents an oxygen atom, Y represents an OH or NH2 group, R1 represents a hydrogen atom or optionally an alkyl group having 1 to 3 carbons, R3 represents a hydrogen atom or a substituted benzyl group, and R4 represents a hydrogen or fluorine atom. These compounds are then powerful inhibitors of PDE2.

Very advantageously, the compounds are chosen from the group consisting of the following compounds:
8-Iodo-4-(N-methyl-N-phenylamino)pyrazolo[1,5-a]-1,3,5-triazine.
8-Iodo-4-[N-methyl-N-(4-nitrophenyl)amino]pyrazolo[1,5-a]-1,3,5-triazine.
8-Iodo-4-(triazol-4-yl)pyrazolo[1,5-a]-1,3,5-triazine.
8-Acetamido-2-methylpyrazolo[1,5-a]-1,3,5-triazin-4-one.
Methyl 4-[(hydroxy) [4-(N-methyl-N-phenylamino)pyrazolo[1,5-a]-1,3,5-triazin-8-yl]methyl]benzoate.
8-[(2-Chlorophenyl)(hydroxy)methyl]-4-(N-methyl-N-phenylamino)-2-n-propylpyrazolo[1,5-a]-1,3,5-triazine.
8-(2-Chlorophenyl)-4-(N-methyl-N-phenylamino)-2-n-propylpyrazolo[1,5-a]-1,3,5-triazine.
8-(2-Chlorophenyl)-4-(N-methylamino)-2-n-propylpyrazolo[1,5-a]-1,3,5-triazine.
Ethyl 3-[4-(N-methyl-N-phenylamino)pyrazolo[1,5-a]-1,3,5-triazin-8-yl]acrylate.
Ethyl 3-[4-(N-methyl-N-phenylamino)pyrazolo[1,5-a]-1,3,5-triazin-8-yl]propionate.
3-[4-(N-Methyl-N-phenylamino)pyrazolo[1,5-a]-1,3,5-triazin-8-yl]propionic acid.
Methyl 4-[[1-oxo-3-[4-(N-methyl-N-phenylamino)pyrazolo[1,5-a]-1,3,5-triazin-8-yl]propyl]amino]benzoate.
4-(Cyclopropylamino)-8-(2-fluorobenzoyl)-2-methylpyrazolo[1,5-a]-1,3,5-triazine.
Ethyl 4-(N-methyl-N-phenylamino)pyrazolo[1,5-a]-1,3,5-triazine-8-carboxylate.
tert-Butyl 3-[4-(N-methyl-N-phenylamino)pyrazolo[1,5-a]-1,3,5-triazin-8-yl]acrylate.
tert-Butyl 3-[4-(N-methyl-N-phenylamino)pyrazolo[1,5-a]-1,3,5-triazin-8-yl]propionate
4-(N-Methyl-N-phenylamino)-8-phenylpyrazolo[1,5-a]-1,3,5-triazine.
4-(N-Methyl-N-phenylamino)-8-(β-D-glycero-pentofuran-3′-ulos-1′-yl)pyrazolo[1,5-a]-1,3,5-triazine.
8-[(3-Furyl)(hydroxy)methyl]-4-(N-methyl-N-phenylamino)-2-n-propylpyrazolo[1,5-a]-1,3,5-triazine.
8-(3-Furylmethyl)-2-n-propyl-4-(N-methyl-N-phenyl-amino)pyrazolo[1,5-a]-1,3,5-triazine.
2-Trifluoromethyl-8-(3-furylmethyl)-4-(cyclopropyl-amino)pyrazolo[1,5-a]-1,3,5-triazine.
2-Thiomethyl-8-(3-furylmethyl)-4-(N-methylamino)pyrazolo[1,5-a]-1,3,5-triazine.
8-(3-Furylmethyl)-4-(N-methylamino)-2-n-propylpyrazolo[1,5-a]-1,3,5-triazine.
2-Trifluoromethyl-8-cyclopentyl-4-(N-methylamino)pyrazolo[1,5-a]-1,3,5-triazine.
2-Pentafluoroethyl-8-(2-methoxybenzyl)-4-(N-methylamino)pyrazolo[1,5-a]-1,3,5-triazine.
4-(N-Cyclopropylamino)-2-trifluoromethyl-8-(2-methoxybenzyl)pyrazolo[1,5-a]-1,3,5-triazine.
4-(N-Cyclopropylamino)-8-(2-methoxybenzyl)-2-n-propyl-pyrazolo[1,5-a]-1,3,5-triazine.
2-Iodo-8-(2-methoxybenzyl)-4-(N-methylamino)pyrazolo[1,5-a]-1,3,5-triazine.
2-Bromo-8-(2-methoxybenzyl)-4-(N-methylamino)pyrazolo[1,5-a]-1,3,5-triazine.
8-[(Hydroxy)(2-thienyl)methyl]-4-(N-methyl-N-phenylamino)-2-n-propylpyrazolo[1,5-a]-1,3,5-triazine.
8-(2-Chlorobenzoyl)-2-trifluoromethyl-4-(N-methylamino)pyrazolo[1,5-a]-1,3,5-triazine.
8-(2-Chlorobenzoyl)-2-pentafluoroethyl-4-(N-methylamino)pyrazolo[1,5-a]-1,3,5-triazine.
8-(2-Chlorobenzoyl)-2-trifluoromethyl-4-(N-cyclopropylamino)pyrazolo[1,5-a]-1,3,5-triazine.
4-(N-Methyl-N-phenylamino)-2-n-propyl-8-(2-thienylmethyl)pyrazolo[1,5-a]-1,3,5-triazine.
4-(N-Methylamino)-2-n-propyl-8-[(2-thienyl)methyl]-pyrazolo[1,5-a]-1,3,5-triazine.
4-(N-Methylamino)-2-trifluoromethyl-8-[(2-thienyl)-methyl]pyrazolo[1,5-a]-1,3,5-triazine.
4-(N-Cyclopropylamino)-2-trifluoromethyl-8-[(2-thienyl)-methyl]pyrazolo[1,5-a]-1,3,5-triazine.
N-[2-(3,4-Dihydroxyphenyl)ethyl]-3-[4-(N-methyl-N-phenylamino)pyrazolo[1,5-a]-1,3,5-triazin-8-yl]propionamide.
3-[4-(N-Methyl-N-phenylamino)pyrazolo[1,5-a]-1,3,5-triazin-8-yl]-N-[3-(2-oxopyrrolidin-1-yl)propyl]propionamide.
N-[2-Hydroxy-2-(3,4-dihydroxyphenyl)ethyl]-3-[4-(N-methyl-N-phenylamino)pyrazolo[1,5-a]-1,3,5-triazin-8-yl]propionamide.
3-(4-Oxopyrazolo[1,5-a]-1,3,5-triazin-8-yl)propionic acid.
Ethyl 3-[4-oxopyrazolo[1,5-a]-1,3,5-triazin-8-yl]acrylate.
Sodium 4-[(hydroxy)[4-oxopyrazolo[1,5-a]-1,3,5-triazin-8-yl]methyl]benzoate.
Sodium 4-[[1-(oxo)-3-(4-oxopyrazolo[1,5-a]-1,3,5-triazin-8-yl)propyl]amino]benzoate.
Sodium 4-[2-(4-oxopyrazolo[1,5-a]-1,3,5-triazin-8-yl)-ethylsulfonylamino]benzoate.
Sodium 4-[1-oxo-3-(2-amino-4-oxopyrazolo[1,5-a]-1,3,5-triazin-8-yl)propylamino]benzoate.
Sodium 4-[1-oxo-3-(2-n-propyl-4-oxopyrazolo[1,5-a]-1,3,5-triazin-8-yl)propylamino]benzoate.
Sodium 4-[1-oxo-3-(2-trifluoromethyl-4-oxopyrazolo[1,5-a]-1,3,5-triazin-8-yl)propylamino]benzoate.
N-[2-(Indol-3-yl)ethyl]-3-(4-oxopyrazolo[1,5-a]-1,3,5-triazin-8-yl)propanamide.
N-[2-(Indol-3-yl)ethyl]-3-(2-amino-4-oxopyrazolo[1,5-a]-1,3,5-triazin-8-yl)propanamide.
N-[1-(Carboxyl)-2-(indol-3-yl)ethyl]-3-(4-oxopyrazolo[1,5-a]-1,3,5-triazin-8-yl)propanamide.
N-[2-(4-Hydroxyphenyl)ethyl]-3-(4-oxopyrazolo[1,5-a]-1,3,5-triazin-8-yl)propanamide.
N-[2-(4-Hydroxyphenyl)ethyl]-3-(2-amino-4-oxopyrazolo[1,5-a]-1,3,5-triazin-8-yl)propanamide.
N-[2-(4-Hydroxyphenyl)ethyl]-3-(2-trifluoromethyl-4-oxopyrazolo[1,5-a]-1,3,5-triazin-8-yl)propanamide.
N-[1-(Carboxyl)-2-(4-hydroxyphenyl)ethyl]-3-(4-oxopyrazolo-[1,5-a]-1,3,5-triazin-8-yl)propanamide.
4-(N-Methyl-N-phenylamino)pyrazolo[1,5-a]-1,3,5-triazine.
2-(4-Methylbenzyl)-8-(2-oxohept-3-yl)pyrazolo[1,5-a]-1,3,5-triazin-4-one.
8-(2-Hydroxy-6-phenylhex-3-yl)-2-(3,4-dimethoxybenzyl)pyrazolo[1,5-a]-1,3,5-triazin-4-one.
Erythro-8-(2-hydroxy-3-nonyl)pyrazolo[1,5-a]-1,3,5-triazin-4-one.
Erythro-4-amino-8-(2-hydroxy-3-nonyl)pyrazolo[1,5-a]-1,3,5-triazine.
Sodium 4-[[3-(1-methyl-4-oxopyrazolo[1,5-a]-1,3,5-triazin-8-yl)-1-(oxo)propyl]amino]benzoate.
8-Benzoyl-2-cyclopropylpyrazolo[1,5-a]-1,3,5-triazin-4-one.
N-[2-(3,4-Dihydroxyphenyl)ethyl]-3-(4-oxopyrazolo[1,5-a]-1,3,5-triazin-8-yl)propionamide.
3-[4-Oxopyrazolo[1,5-a]-1,3,5-triazin-8-yl)-N-[3-(2-oxo-pyrrolidin-1-yl)propyl]propionamide.
N-[2-Hydroxy-2-(3,4-dihydroxyphenyl)ethyl]-3-[4-oxopyrazolo-[1,5-a]-1,3,5-triazin-8-yl]propionamide.
8-(2′-Deoxy-β-D-ribofuranosyl)-4-(N-methyl-N-phenyl-amino)pyrazolo[1,5-a]-1,3,5-triazine.
8-(2′-Deoxy-β-D-ribofuranosyl)-4-[N-methyl-N-(4-nitro-phenylamino)]pyrazolo[1,5-a]-1,3,5-triazine.
8-(2′-Deoxy-β-D-xylofuranosyl)-4-(N-methyl-N-phenylamino)pyrazolo[1,5-a]-1,3,5-triazine.
8-(2′-Deoxy-β-D-xylofuranosyl)-4-[N-methyl-N-(4-nitro-phenylamino)]pyrazolo[1,5-a]-1,3,5-triazine.
4-Amino-8-(2′-deoxy-β-D-ribofuranosyl)pyrazolo[1,5-a]-1,3,5-triazine.
8-(2′-Deoxy-β-D-ribofuranosyl)pyrazolo[1,5-a]-1,3,5-triazin-4-one.
4-Amino-8-(2′-deoxy-β-D-xylofuranosyl)pyrazolo[1,5-a]-1,3,5-triazine.
8-(2′-Deoxy-β-D-xylofuranosyl)pyrazolo[1,5-a]-1,3,5-triazin-4-one.
4-Amino-2-fluoro-8-[trans-2,trans-3-dihydroxy-4-(hydroxymethyl)cyclopent-4-enyl]pyrazolo[1,5-a]-1,3,5-triazine.
4-Amino-8-[trans-2,trans-3-dihydroxy-4-(hydroxymethyl)-cyclopent-4-enyl]pyrazolo[1,5-a]-1,3,5-triazine.
2-Fluoro-8-[trans-2,trans-3-dihydroxy-4-(hydroxymethyl)cyclopent-4-enyl]pyrazolo[1,5-a]-1,3,5-triazin-4-one.
8-[trans-2,trans-3-dihydroxy-4-(hydroxymethyl)cyclopent-4-enyl]pyrazolo[1,5-a]-1,3,5-triazin-4-one.
(1S,4R)-2-Amino-4-(cyclopropylamino)-8-[4-(hydroxymethyl)cyclopent-2-en-1-yl]pyrazolo[1,5-a]-1,3,5-triazine.
cis-2-Amino-4-(cyclopropylamino)-8-[4-(hydroxymethyl)-cyclopent-2-en-1-yl]pyrazolo[1,5-a]-1,3,5-triazine.
4-Amino-7-chloro-8-(β-D-ribofuranosyl)pyrazolo[1,5-a]-1,3,5-triazine-3′,5′-cyclophosphate.
bis-(2,2,2-Trifluoroethyl) [2-[2-amino-4-(4-methoxyphenylthio)pyrazolo[1,5-a]-1,3,5-triazin-8-yl]ethoxy]-methylphosphonate.
4-Amino-8-(3′-deoxy-β-D-ribofuranosyl)pyrazolo[1,5-a]-1,3,5-triazine.
8-(3′-Deoxy-β-D-ribofuranosyl)pyrazolo[1,5-a]-1,3,5-triazin-4-one.
2-Amino-8-(3′-deoxy-β-D-ribofuranosyl)pyrazolo[1,5-a]-1,3,5-triazin-4-one.
4-Amino-2-chloro-8-(2′-deoxy-β-D-ribofuranosyl)pyrazolo[1,5-a]-1,3,5-triazine.
cis-2-Amino-4-(cyclopropylamino)-8-[2-(hydroxymethyl)-1,3-dioxolan-4-yl]pyrazolo[1,5-a]-1,3,5-triazine.
4-Amino-8-(2′,3′-dideoxy-2′-fluoro-β-D-ribofuranosyl)-pyrazolo[1,5-a]-1,3,5-triazine.
4-Amino-8-(2′,3′-dideoxy-2′-fluoroarabinosyl)pyrazolo[1,5-a]-1,3,5-triazine.
2-Amino-8-[4-acetyloxy-3-(acetyloxymethyl)butyl]pyrazolo[1,5-a]-1,3,5-triazine.
4-Amino-2-chloro-8-(2′-deoxy-2′-fluoro-β-D-ribofuranosyl)pyrazolo[1,5-a]-1,3,5-triazine.
4-Amino-8-(2′-deoxy-2′-fluoro-β-D-ribofuranosyl)pyrazolo[1,5-a]-1,3,5-triazine.
8-(2′-Deoxy-2′-fluoro-β-D-ribofuranosyl)pyrazolo[1,5-a]-1,3,5-triazin-4-one.
S-[[4-Amino-8-(5′-deoxy-β-D-ribofuranosyl)pyrazolo[1,5-a]-1,3,5-triazine]-5′-yl]methionine (bioisostere of S-adenosylmethionine).
2-Amino-4-[(4-bromo-2-thienyl)methoxy]pyrazolo[1,5-a]-1,3,5-triazine.
(R)-4-Benzylamino-2-[1-(hydroxymethyl)propylamino]-8-isopropylpyrazolo[1,5-a]-1,3,5-triazine.
(S)-4-Benzylamino-2-[1-(hydroxymethyl)propylamino]-8-isopropylpyrazolo[1,5-a]-1,3,5-triazine.
2′-(Butyryl)-4-(N-butyrylamino)-8-(β-D-ribofuranosyl)-pyrazolo[1,5-a]-1,3,5-triazine-3′,5′-cyclophosphate.
cis-2,4-Diamino-8-[2-(hydroxymethyl)-1,3-dioxolan-4-yl]pyrazolo[1,5-a]-1,3,5-triazine.
cis-2-Amino-8-[2-(hydroxymethyl)-1,3-dioxolan-4-yl]-pyrazolo[1,5-a]-1,3,5-triazin-4-one.
cis-8-[2-(Hydroxymethyl)-1,3-dioxolan-4-yl]pyrazolo[1,5-a]-1,3,5-triazin-4-one.
cis-4-Amino-8-[2-(hydroxymethyl)-1,3-dioxolan-4-yl]-pyrazolo[1,5-a]-1,3,5-triazine.
(1′S,2′R)-2-Amino-8-[[1′,2′-bis(hydroxymethyl)cycloprop-1′-yl]methyl]pyrazolo[1,5-a]-1,3,5-triazin-4-one.
(1′S,2′R)-8-[[1′,2′-bis(Hydroxymethyl)cycloprop-1′-yl]-methyl]pyrazolo[1,5-a]-1,3,5-triazin-4-one.
(1′S,2′R)-4-Amino-8-[[1′,2′-bis(hydroxymethyl)cycloprop-1′-yl]methyl]pyrazolo[1,5-a]-1,3,5-triazine.
2-Amino-8-[(2-hydroxyethoxy)methyl]pyrazolo[1,5-a]-1,3,5-triazin-4-one.
8-[(2-Hydroxyethoxy)methyl]pyrazolo[1,5-a]-1,3,5-triazin-4-one.
4-Amino-8-[(2-hydroxyethoxy)methyl]pyrazolo[1,5-a]-1,3,5-triazine.
2-Amino-8-[4-hydroxy-3-(hydroxymethyl)butyl]pyrazolo[1,5-a]-1,3,5-triazin-4-one.
4-Amino-8-[4-hydroxy-3-(hydroxymethyl)butyl]pyrazolo[1,5-a]-1,3,5-triazine.
8-[4-Hydroxy-3-(hydroxymethyl)butyl]pyrazolo[1,5-a]-1,3,5-triazin-4-one.
2-Amino-8-[2-hydroxy-1-(hydroxymethyl)ethoxymethyl]-pyrazolo[1,5-a]-1,3,5-triazin-4-one.
8-[2-Hydroxy-1-(hydroxymethyl)ethoxymethyl]pyrazolo[1,5-a]-1,3,5-triazin-4-one.
4-Amino-8-[2-hydroxy-1-(hydroxymethyl)ethoxymethyl]-pyrazolo[1,5-a]-1,3,5-triazine.
2-[(2-Amino-4-oxopyrazolo[1,5-a]-1,3,5-triazin-8-yl)-methoxy]ethyl valinate.
8-(2′,3′-Dideoxy-β-D-ribofuranosyl)pyrazolo[1,5-a]-1,3,5-triazin-4-one.
8-(2′,3′-Dideoxy-2′,2′-difluoro-β-D-ribofuranosyl)-pyrazolo[1,5-a]-1,3,5-triazin-4-one.
8-(2′-Deoxy-β-D-ribofuranosyl)pyrazolo[1,5-a]-1,3,5-triazin-4-one.
bis(Pivaloyloxymethyl) [2-(4-aminopyrazolo[1,5-a]-1,3,5-triazin-8-yl)ethoxy]methylphosphonate.
Sodium [2-(4-aminopyrazolo[1,5-a]-1,3,5-triazin-8-yl)-ethoxy]methylphosphonate.
4-Amino-8-[2-[[bis(pivaloyloxymethyl)phosphonyl]-methoxy]ethyl]pyrazolo[1,5-a]-1,3,5-triazine.
cis-8-[2-(Hydroxymethyl)-1,3-oxathiolan-5-yl]pyrazolo[1,5-a]-1,3,5-triazin-4-one.
cis-8-[2-(Hydroxymethyl)-1,3-oxathiolan-5-yl]-2-oxo-pyrazolo[1,5-a]-1,3,5-triazin-4-one.
cis-8-[2-(Hydroxymethyl)-1,3-oxathiolan-5-yl]-2-thioxo-pyrazolo[1,5-a]-1,3,5-triazin-4-one.
cis-2-Amino-8-[2-(hydroxymethyl)-1,3-oxathiolan-5-yl]pyrazolo[1,5-a]-1,3,5-triazin-4-one.
cis-4-Amino-8-[2-(hydroxymethyl)-1,3-oxathiolan-5-yl]-pyrazolo[1,5-a]-1,3,5-triazine.
8-[[3R,4R)-3-Hydroxy-4-(hydroxymethyl)pyrrolidin-1-yl]-methyl]pyrazolo[1,5-a]-1,3,5-triazin-4-one.
4-Amino-8-[[(3R,4R)-3-hydroxy-4-(hydroxymethyl)pyrrolidin-1-yl]methyl]pyrazolo[1,5-a]-1,3,5-triazine.

The compounds of the invention may be in the form of salts, in particular of base or acid addition salts, preferably compatible with pharmaceutical use. Among pharmaceutically acceptable acids, mention may be made, without implied limitation, of hydrochloric acid, hydrobromic acid, sulfuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulfonic or ethanesulfonic acid, camphoric acid, etc. Among pharmaceutically acceptable bases, mention may be made, without implied limitation, of sodium hydroxide, potassium hydroxide, triethylamine, tert-butylamine, etc.

The compounds of the invention may also have one or more asymmetric center(s) and may be isolated in optically active form or in the form of their racemic mixture. Methods for obtaining optically active forms, for example by resolution of a racemic form or by synthesis using racemic starting products, are well known to those skilled in the art. Similarly, the geometric isomers of olefins or of double bonds of C═N type can be isolated and characterized in cis or trans form or can be used in the form of a cis and trans mixture.

According to the invention, at least one of the atoms of the molecules described can be replaced with an isotope (an atom which has the same atomic number but a different mass). Mention may be made, without implied limitation, of the example of the isotopes of the hydrogen atom, tritium and deuterium, and also those of carbon, C-13 and C-14.

According to the invention, the term “alkyl” denotes a linear or branched hydrocarbon-based radical having advantageously from 1 to 6 carbon atoms, such as methyl, ethyl, propyl, isopropyl, butyl, isobutyl, tert-butyl, pentyl, neopentyl or n-hexyl. C1-C4 groups are preferred. The alkyl groups can be substituted with an aryl group as defined hereinafter; in which case, this is described as an arylalkyl group. Examples of arylalkyl groups are in particular benzyl and phenethyl.

The term “cycloalkyl” denotes a cyclic hydrocarbon-based system which may comprise advantageously from 3 to 6 carbon atoms and may be monocyclic or polycyclic. Mention may in particular be made of the cyclopropyl and cyclohexyl groups.

The “alkenyl” groups are linear, branched or cyclic hydrocarbon-based radicals containing one or more double bonds. They contain advantageously from 2 to 6 carbon atoms, and preferably one or two double bonds. The alkenyl groups can be substituted with an aryl group as defined hereinafter; in which case, this is described as an arylalkenyl group.

The “alkynyl” groups are linear or branched hydrocarbon-based radicals containing one or more triple bonds. They contain advantageously from 2 to 6 carbon atoms, and preferably one or two triple bonds. The alkynyl groups can be substituted with an aryl group as defined hereinafter; in which case, this is called an arylalkynyl group.

The “alkoxy” groups correspond to the alkyl and cycloalkyl groups defined above linked to the nucleus via an —O— (ether) bond. Methoxy, ethoxy, n-propyloxy, i-propyloxy, n-butoxy, s-butoxy, t-butoxy, n-pentoxy and s-pentoxy groups are most particularly preferred.

The “acyl” groups correspond to the alkyl, cyclo-alkyl and aryl groups defined above connected to the nucleus via a —CO bond. As an example of acyl groups, mention may in particular be made of acetyl, propionyl, cyclohexylcarbonyl and benzoyl groups.

The “aryl” groups are monocyclic, bicyclic or tricyclic aromatic hydrocarbon-based systems, preferably monocyclic or bicyclic aromatic hydrocarbon-based systems having from 6 to 18 carbon atoms, even more preferably 6 carbon atoms. Mention may be made, for example, of phenyl, naphthyl and biphenyl groups.

The “heteroaryl” groups denote aromatic hydrocarbon-based systems as defined above comprising one or more cyclic hetero atoms. They are preferably cyclic aromatic hydrocarbon-based systems containing from 5 to 18 carbon atoms and one or more cyclic hetero atoms, in particular from 1 to 4 cyclic hetero atoms chosen from N, O or S. Among the preferred heteroaryl groups, mention may in particular be made of benzothienyl, benzofuryl, pyrrolidinyl, thiazolyl, thienyl, furyl, pyranyl, pyrrolyl, 2H-pyrrolyl, imidazolyl, benzimidazolyl, pyrazolyl, isothiazolyl, isoxazolyl and indolyl groups, this list not being limiting.

The aryl and heteroaryl groups can be substituted with an alkyl, alkenyl or alkynyl group as defined above. In the case of an aryl or of a heteroaryl substituted with an alkyl group, this is referred to as an alkylaryl group. Examples of alkylaryl groups are in particular tolyl, mesityl and xylyl. In the case of an aryl or of a heteroaryl substituted with an alkenyl group, this is referred to as an alkenylaryl group. An example of an alkenylaryl group is in particular the cinnamyl group. In the case of an aryl or of a heteroaryl substituted with an alkynyl group, this is referred to as an alkynylaryl group.

The “heterocycles” denote aromatic or nonaromatic hydrocarbon-based systems comprising one or more cyclic hetero atoms. They are preferably cyclic hydrocarbon-based systems containing from 5 to 18 carbon atoms and one or more cyclic hetero atoms, in particular from 1 to 4 cyclic hetero atoms chosen from N, O or S. Among the preferred heterocycles, mention may in particular be made of morpholine, piperazine, piperidine, tetrahydrofuran, oxazolidine and isoxazoline, this list not being limiting.

The term “halogen” is intended to mean a fluorine, chlorine, bromine or iodine atom.

The term “hetero atom” is intended to mean an atom chosen from O, N and S.

The compounds according to the invention are capable in particular of increasing the synthesis and/or the release of neurotrophic factors.

Among the growth factors induced by the administration of the novel derivatives, mention may in particular be made, without implied limitation, of: NGF (nerve growth factor), NT-3, BDNF (brain-derived neurotrophic factor), ciliary neurotrophic factor (CNTF), bFGF (basic fibroblast growth factor), neurotrophin-3, protein S-100 beta (Rathbone, M. P. et al. Prog. Neurobiol. (1999), 59, 663-690), and also other neurotrophic factors involved in the survival and in the regeneration of sensory or motor neurons. This increase in the synthesis and/or in the release of neurotrophic factor(s) is the result of a modulation of carbon monoxide-dependent guanylate cyclase and/or of the inhibition of a phosphodiesterase. In both cases, an increase in intracellular cGMP levels will be observed.

The compounds according to the invention can act on either enzyme (guanylate cyclase or phosphodiesterase) or can combine a simultaneous action on these two targets. In the latter case, a synergistic action will be obtained and will result in a large intracellular increase in cGMP, possibly combined with an increase in cAMP. For certain states or certain pathologies, a mixed phosphodiesterase inhibitor, i.e. an inhibitor that acts at the same time on at least two different families of phosphodiesterase (in particular PDE2 and PDE4), will be preferred. For example, an inhibitor of phosphodiesterase type 4 (PDE4) will make it possible to treat the inflammatory component relating to the states or pathologies targeted. This anti-inflammatory effect is in particular the result of a large dose-dependent decrease in the production of tumor necrosis factor alpha (TNF-a) by the pro-inflammatory cells. Moreover, an inhibitor of PDE4 will also make it possible to treat depression, dementia or alternatively anxiety.

Certain molecules according to the invention are powerful and selective inhibitors of phosphodiesterase type 4 (PDE4), which can act possibly simultaneously on the increase in synthesis and in release of one or more neurotrophic factors. These PDE4 inhibitors have demonstrated a marked anti-inflammatory effect which can advantageously be used for treating and preventing inflammatory and autoimmune diseases. The PDE4 inhibitors (PDE4Is) are particularly advantageous for the treatment of asthma and of chronic obstructive bronchopathies, but also of other conditions such as rhinitis, acute respiratory stress syndrome, allergies, dermatitis, psoriasis, rheumatoid arthritis, multiple scleroses (in particular multiple sclerosis), dyskinesias, glomerulonephritis, osteoarthritis, cancer, septic shock, AIDS, Crohn's disease, osteoporosis, rheumatoid arthritis or obesity. The PDE4Is also have central effects that are particularly advantageous for the treatment of depression, of anxiety, of schizophrenia, of bipolar disorder, of attention deficits, of fibromyalgia, of Parkinson's disease and Alzheimer's disease, of amyotrophic sclerosis, of multiple scleroses, of Lewy body dementias and of other psychiatric disorders. The novel PDE4 inhibitors are advantageously devoid of any emetic or hypotensive effect.

Certain compounds of the invention advantageously have anti-inflammatory effects, immunomodulatory, neurological, antimicrobial or antiviral properties, or cardiovascular effects. These properties combined with the main activity may be due to a pharmacophore that is different from that which makes it possible to engender the main property. The combination of these two properties within the same molecule is particularly advantageous for the treatment of Alzheimer's disease and Parkinson's disease, of AIDS, of diabetes, and also of memory disorders, in particular those associated with senescence. In certain cases, an inhibitory property with respect to PDE, cyclin-dependent kinases, monoamine oxygenase or the “multidrug” transporter will make it possible to obtain these combined properties.

The compounds according to the invention also advantageously have an excellent central tropism and are advantageously devoid of any hyperalgic and pro-inflammatory effects. Other compounds are advantageously devoid of central effects and penetrate the central nervous system very poorly.

The invention also relates to the methods for preparing the compounds of formula (I).

The compounds of the invention can be prepared from commercial products, by using a combination of chemical reactions known to those skilled in the art.

In this regard, according to a first method, the compounds of general formula (Ib) according to the invention in which Y is different from chlorine and from bromine can be obtained from a compound of formula (Ib) in which Y is a chlorine or bromine atom, using the following methods:

1. When Y in the formula of the final product (Ib) is a group NRxRy, by reaction with an amine of formula HNRxRy, in an organic solvent at ambient temperature. As solvent, mention may in particular be made of dichloromethane or dimethylformamide.

2. When Y in the formula of the final product (Ib) is a (C₁-C₆)alkyl group, by reaction with a compound of formula YLi, in an anhydrous solvent at a temperature of between −80 and −20° C., preferably in the region of −78° C. As solvent, mention maybe made of ethers, in particular tetrahydrofuran.

3. When Y in the formula of the final product (Ib) is a (C1-C6)alkyn-1-yl group, by reaction with a compound of formula YH, in which Y is a true acetylenic group, in the presence of copper iodide, of palladium chloride, of triphenylphosphine and of a base, for example triethylamine. As solvent, use may in particular be made of acetonitrile; the reaction is preferably carried out at ambient temperature.

4. When Y in the formula of the final product (Ib) is a (C6-C12)aryl group, by reaction with an aromatic compound, for example N,N-dimethylaniline, at a temperature of between 80 and 130° C., preferably in the region of 120° C. and in a sealed tube. As solvent, use is preferably made of a polar aprotic solvent, for example chloroform. These compounds can be obtained by coupling with palladium using, for example, a boronic acid in the presence of a base, for example sodium bicarbonate.

5. When Y in the formula of the final product (Ib) is a group ORx, by reaction with an alcohol of formula HORx at ambient temperature. If Rx is OH, the alcohol will be replaced in this reaction with water or a hydroxide, for example sodium hydroxide.

6. When Y in the formula of the final product (Ib) is a group SRx, by reaction with a thiol of formula RxSH. As solvent, mention may in particular be made of tetrahydrofuran.

7. The compounds where Y in the formula of the final product (Ib) is an SH group can be obtained directly by treating the compounds where Y is an OH group with Lawesson's reagent.

The compounds of general formula (Ib) according to the invention in which Y is different from chlorine can also be obtained from a compound of formula (Ib) in which Y is a particular group NRxRy, for example an N-methyl-N-phenylamino, N-methyl-N-(4-nitrophenyl)amino, N-methyl-N-(4-acylaminophenyl)amino or triazole group, using the following methods:

1. When Y in the formula of the final product is a group NRxRy, by reaction with an amine of formula HNRxRy, in a protic solvent at a temperature of between 10° C. and 130° C., preferably in the region of 90° C., in a sealed tube. As solvent, mention may in particular be made of methanol or ethanol.

2. When Y in the formula of the final product (Ib) is a hydroxyl group (OH), by reaction with a hydroxide, for example sodium hydroxide, in a protic solvent at a temperature of between −10 and 100° C., preferably in the region of 25° C. As solvent, mention may be made of alcohols, or alcohol-water mixtures, in particular ethanol or an ethanol-water mixture.

The compounds of general formula (I) according to the invention in which R1 represents a (C1-C12)alkyl group can be prepared from the compounds of general formula (I) where R1 is H, by means of an alkylation reaction using a base, and an alkylating agent. As a base, mention may in particular be made of potassium carbonate and sodium hydride. The preferred alkylating agents are halides or epoxides. The presence of a phase transfer catalyst makes it possible, according to the case, to improve the reaction yields.

The compounds of general formula (I) in which X═S according to the invention can be obtained from a compound of formula (I) in which X═O, by means of a reaction using Lawesson's reagent in an organic solvent, for example toluene.

The compounds of general formulae (Ia) and (Ib) according to the invention in which R1=H can be prepared by means of a method comprising the following steps:

a) reaction of a compound of general formula (V)

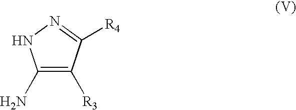

in which R₃ and R₄ are as defined above;
with a compound comprising a group of formula R₂C(GP)═NH, in which R₂ is as defined above and GP represents a leaving group, for example a halogen atom, a (C₁-C₄)alkoxy group or a thio(C₁-C₄)alkyl group, so as to obtain a compound of formula (VI)

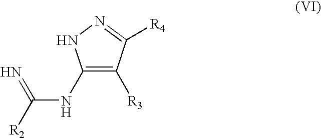

in which R₂, R₃ and R₄ are as defined above;

b) reaction of the compound of formula (VI) with a dielectrophile, for example diethyl carbonate or an orthoester, so as to obtain a compound of formula (Ia) or (Ib) in which R2, R3, R4, X and Y are as defined above and R1 is H.

The compound comprising a group of formula R2C(GP)═NH in step a) is preferably an imidate of formula R2(OMe)═NH.HCl, in which R2 is as defined above. The reaction is advantageously carried out in the presence of a base, for example sodium acetate, in an inert solvent at ambient temperature. As solvent, mention may be made of acetonitrile. At the end of the reaction, the product is in this case obtained in the form of an acetate.

Step b) is advantageously carried out in the presence of a base, for example sodium ethanolate, at a temperature of between 20 and 150° C., preferably in the region of 100° C., when the dielectrophile used is ethyl carbonate, for a period of between 3 and 48 hours, preferably of around 24 hours. In this case, a compound of general formula (VII) is obtained, in which R₂, R₃ and R₄ are as defined above.

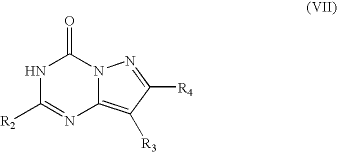

According to another variant of the invention, the compounds of general formula (Ib) according to the invention can be obtained from a compound of formula (VII), in which R2, R3 and R4 are as defined above, using the following methods:

1. When Y in the formula of the final product is a group NRxRy, by reaction with phosphorus oxychloride (POCl₃) and a tertiary amine, for example N,N-dimethylaniline, in an aprotic solvent at a temperature of between 60° C. and 140° C., so as to obtain a compound of formula (VIII)

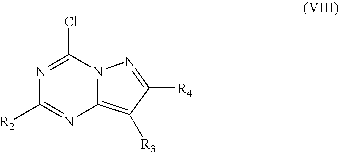

in which R₂, R₃ and R₄ are as defined above. This compound (VIII) can be isolated or directly converted into a compound of general formula (Ib) in which Y is a group NRₓR_{y}, by reaction with an amine of formula HNRₓR_{y}, at ambient temperature.

2. When Y in the formula of the final product is a group NPhCH3 by reaction with phosphorus oxychloride (POCl3) and N,N-dimethylaniline in an aprotic solvent at a temperature of between 60° C. and 140° C., so as to obtain a compound of formula (IX)

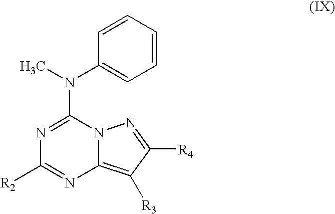

in which R₂, R₃ and R₄ are as defined above.

3. When Y in the formula of the final product (Ib) is an SH group, by reaction with Lawesson's reagent in an aprotic solvent.

According to another variant of the invention, the compounds of general formula (Ib) according to the invention can be obtained from a compound of formula (IX) using the following methods:

1. When Y in the formula of the final product (Ib) is a group NRxRy, by reaction with an amine of formula HNRxRy, in a protic solvent, at a temperature of between 20° C. and 130° C., preferably in the region of 100° C. As solvent, mention may be made of ethanol.

2. When Y in the formula of the final product (Ib) is an OH group, by reaction with a hydroxide, for example sodium hydroxide, in a protic solvent, at a temperature of between 20° C. and 130° C., preferably in the region of 100° C. As solvent, mention may be made of ethanol.

3. When R3 in the formula of the final product (Ib) is an acyl group, by reaction of an acid chloride, preferably in the presence of a Lewis acid, at a temperature of between 20° C. and 80° C., preferably in the region of 60° C., with a compound of formula (IX) in which R3 is a hydrogen atom. This reaction is advantageously carried out in the absence of solvent. Among Lewis acids, mention may in particular be made of tin(IV) chloride.

4. When R3 in the formula of the final product (Ib) is a nitro group, by reaction of nitric acid, preferably in a protic medium. In this case, a product of general formula (X) is predominantly obtained

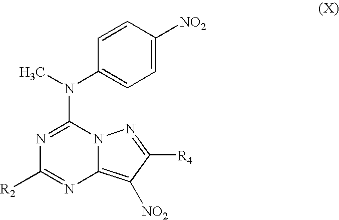

in which R₂ and R₄ are as defined above.

According to another variant of the invention, the compounds of general formula (Ib) according to the invention can be obtained from a compound of formula (X) by means of a method comprising the following steps:

1. Catalytic hydrogenation, for example in the presence of palladium-on-charcoal.

A compound of general formula (XI) is then obtained

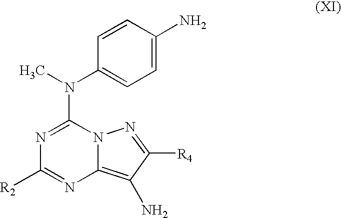

in which R₂ and R₄ are as defined above.

2. Acylation of a compound of general structure (XI) using an acylating agent, of general formula acyl-GP where GP has the same meanings as above. As acylating agent, mention may be made of acid chlorides. This reaction is advantageously carried out in an organic solvent in the presence of a base. As base, mention may be made of triethylamine and as solvent, dichloromethane. A compound of general formula (XII) is then obtained

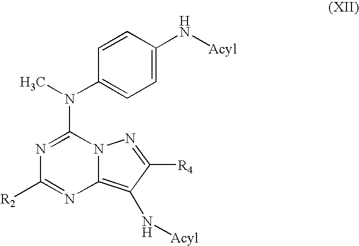

in which R₂ and R₄ are as defined above.

3. The compound of general formula (XII) is converted into compounds of general formula (Ib) according to the invention by the action of a nucleophile of general formula YH or Y—, in which Y is as defined above. Y can for example be an amine of the type HNRxRy, or the hydroxide anion.

According to another variant of the invention, the compounds of general formula (Ib) according to the invention can be obtained from a compound of formula (XIII)

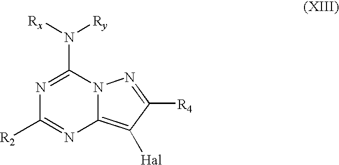

in which Rₓ, R_{y}, R₂ and R₄ are as defined above and Hal represents a halogen atom, preferably an iodine atom, using the following methods:

1. A coupling reaction with palladium, in the presence of a boronic acid or of an alkene or of an alkyne or of any other reagent conventionally used in this type of coupling reaction, at a temperature of between 10 and 130° C.

2. By the action of a strong base, for example n-butyllithium, at a temperature of between −20° C. and −80° C., preferably at −78° C. A carbanion is then obtained in the 8-position of the pyrazolo[1,5-a]-1,3,5-triazine. This carbanion can then be coupled with various electrophilic agents. Aldehydes will be preferred as electrophilic agents.

According to another variant of the invention, the compounds of general formula (Ia) or (Ib) where R3 is an acyl group can be obtained according to the invention from a compound of formula (Ia) or (Ib) in which R3 is a hydrogen atom, by reaction of an acid chloride, preferably in the presence of a Lewis acid, at a temperature of between 20° C. and 80° C., preferably in the region of 60° C., with a compound of formula (IX) in which R3 is a hydrogen atom. This reaction is advantageously carried out in the absence of solvent. Among Lewis acids, mention may in particular be made of tin(IV) chloride.

The compounds of general formula (VII) can be prepared by reaction of a compound of general formula (XIV)

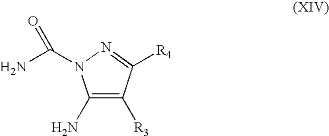

in which R₃ and R₄ are as defined above, with a compound comprising an electrophilic agent, for example an orthoester, at a temperature between 10 and 140° C., preferably in the region of 100° C.

A subject of the invention is also a pharmaceutical composition comprising at least one compound of formula (I) and a pharmaceutically acceptable vehicle or excipient.

A subject of the invention is also the use of at least one compound of formula (I), for producing a medicinal product intended to treat or prevent a human or animal disease for which an increase in the synthesis and/or the release of neurotrophic factors is desired.

A subject of the invention is also the use of at least one compound of formula (I), for producing a medicinal product intended to treat or prevent a human or animal disease for which an inhibition of at least one cyclic nucleotide phosphodiesterase chosen from PDE2 and PDE4 is desired. The PDE4 inhibitors are advantageously devoid of any emetic effect and may advantageously be selective with respect to a subtype of PDE4 chosen from PDE4A-D.

The invention relates more particularly to the use of the compounds of formula (I), for producing a medicinal product intended to treat or prevent pathologies involving neuronal degeneration.

Thus, the pharmaceutical compositions containing the compounds according to the invention, in particular the substituted pyrazolo[1,5-a]-1,3,5-triazines, can be used in the treatment of neurodegenerative or neurological disorders of the central and peripheral systems, including cognitive disorders related to age, such as senility and Alzheimer's disease, nerve lesions, prion diseases (in particular spongiform encephalopathies of the Creutzfeldt-Jakob disease type), peripheral neuropathies, including neuropathies associated with the administration of drugs (oncolytics, etc.), Down's syndrome, cerebral strokes and conditions with spasms such as epilepsy. The compounds according to the invention are particularly advantageous in the treatment of pathologies or of states in which the central or peripheral neuronal functions are impaired, and more particularly in states or diseases resulting from excessive neuronal death, such as neurodegenerative or neurological disorders of the central and peripheral systems of chronic or acute nature. Mention may in particular be made, without implied limitation, of cognitive and mental disorders related to age (in particular senility), Alzheimer's disease, Parkinson's disease, amyotrophic lateral sclerosis, Down's syndrome, multiple scleroses, Huntington's disease, cerebral strokes, peripheral neuropathies (including drug-related neuropathies or diabetes-related neuropathies), retinopathies (in particular pigmentary retinitis), traumas (accidents to the vertebral column, compression of the optic nerve subsequent to a glaucoma and, in general, any central or peripheral nerve lesion, etc.), or neuronal disorders caused by the action of chemical products, and also disorders associated with these states or diseases which may be disorders that are secondary to the primary pathology. In many cited cases, it is most commonly the progressive death of motoneurons and/or sensory neurons which will be the cause of the disorders observed. In certain cases, the pharmaceutical compositions containing the compounds according to the invention, in particular the substituted pyrazolotriazines, may be devoid of any neurotrophic effect but may act strongly as an inhibitor of PDE2 or of PDE4 or may combine a simultaneous action on these two enzymes (mixed PDE2/PDE4 inhibitor). These compounds are particularly advantageous for the treatment of inflammatory and autoimmune diseases.

This treatment may also be administered in a preventive capacity, to patients in whom there is a risk of these same diseases developing.

Certain compounds of the invention have anti-inflammatory effects, immunomodulatory, neurological, antimicrobial or antiviral properties, or alternatively cardiovascular effects. The combination of these two properties within the same molecule is particularly advantageous for the treatment of Alzheimer's and Parkinson's disease, of AIDS, and also of memory disorders, in particular those associated with senescence.

The compounds of the invention are also particularly advantageous for the treatment of central nervous system pathologies, such as more specifically depression, schizophrenia, bipolar disorder, attention deficit disorders, conditions with spasms such as epilepsy, fibromyalgia, or Lewy body dementia.

For the purpose of the invention, the term “treatment” denotes both a preventive and curative treatment, which may be used alone or in combination with other agents or treatments. In addition, it may involve a treatment of chronic or acute disorders.

The compounds or compositions according to the invention may be administered in various ways and in various forms. Thus, they may be administered by injection or orally, for instance intravenously, intramuscularly, subcutaneously, transdermally, intra-arterially, etc., intravenous, intramuscular, subcutaneous and oral administrations being preferred. For injections, the compounds are generally packaged in the form of liquid suspensions which can be injected by means of syringes or of infusions, for example. In this regard, the compounds are generally dissolved in saline, physiological, isotonic, buffered, etc. solutions that are compatible with pharmaceutical use and are known to those skilled in the art. Thus, the compositions may contain one or more agents or vehicles chosen from dispersing agents, solubilizing agents, stabilizing agents, preserving agents, etc. Agents or vehicles that can be used in liquid and/or injectable formulations are in particular methylcellulose, hydroxymethylcellulose, carboxymethylcellulose, polysorbate 80, mannitol, gelatin, lactose, plant oils, acacia, etc.

The compounds may also be administered in the form of gels, oils, tablets, eye lotions, suppositories, powders, gelatin capsules, capsules, etc., optionally by means of pharmaceutical forms or of devices that ensure prolonged and/or delayed release. For this type of formulation, an agent such as cellulose, carbonates or starches is advantageously used.

It is understood that the flow rate and/or the dose injected can be adjusted by those skilled in the art as a function of the patient, of the pathology concerned, of the mode of administration, etc.

Typically, the compounds are administered at doses that can range between 0.1 μg and 100 mg/kg of body weight, more generally from 0.01 to 50 mg/kg, typically between 0.1 and 50 mg/kg. In addition, repeat injections can, where appropriate, be given. Furthermore, for chronic treatments, delayed or prolonged systems may be advantageous.

The invention is illustrated by means of the examples and the FIGURE which follow, which should be considered as nonlimiting illustrations.

Examples 1 to 3 concern the chemical synthesis and examples 4-7 illustrate the pharmacological activity of the compounds of the invention.

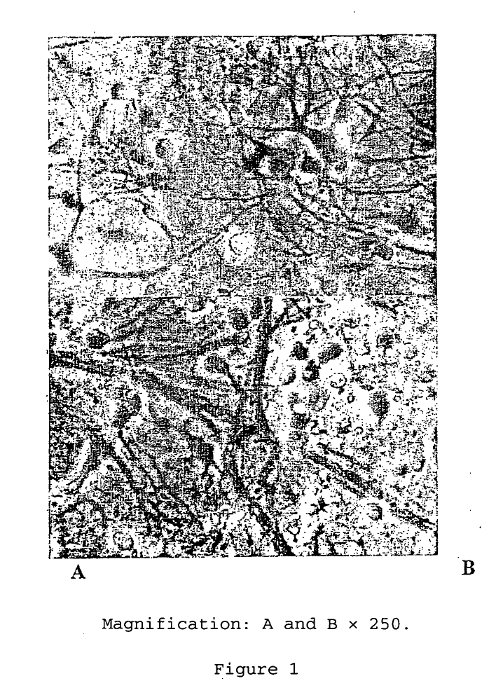

FIG. 1 represents the effect of the molecule Ia5 on neurons in culture. The neurons are cultured in Neurobasal medium from fetal rat brain cortex according to the procedure described in example 4 and are photographed without staining 17 days after being placed in culture. Culture A is a control culture without compound. The molecule Ia5 was added to culture B on the 8th day after the placing in culture, at a concentration of 50 μM.

### EXAMPLE 1

### Synthesis of the Compounds of Formulae VI-XIII (Synthesis Intermediates)

The starting products are commercially available or can be synthesized by conventional methods known to those skilled in the art.

N-(pyrazol-3-yl)acetamidine acetate.NaCl (VIa). 516 mg of NaOAc are added, under argon, to a solution of 500 mg of 3-aminopyrazole and of 692 mg of methyl iminoacetate hydrochloride in 10 ml of CH₃CN. The mixture is stirred at ambient temperature for 12 hours. It is filtered and washed twice with 2 ml of CH₃CN and twice with 5 ml of Et₂O. 1.34 g of a white powder are obtained, yield: 92%. Mp: 159° C. ¹H-NMR (300 MHz, DMSO-d₆): 1.89 (s, 3H, CH₃), 1.98 (s, 3H, CH₃), 5.86 (s, 1H pyrazole), 7.54 (s, 1H pyrazole).

N-(pyrazol-3-yl)trifluoroacetamidine acetate (VIb). 3.4 g of S-p-chlorophenyltrifluorothioacetimidate are added, under argon, to a solution of 1.18 g of 3-aminopyrazole in 15 ml of CH₃CN. After 5 minutes, 812 μl of AcOH are added dropwise. After 8 hours, the mixture is evaporated to dryness. 5 ml of Et₂O and 30 ml of hexane are added. The mixture is stirred vigorously for 30 minutes. It is then filtered and washed twice with 5 ml of hexane and then twice with 5 ml of H₂O. M: 178.12. Yield=93%. Mp: 132° C. ¹H-NMR (200 MHz, CDCl₃): 6.38 (d, J=2.4, 1H pyrazole), 7.51 (d, J=2.4, 1H pyrazole).

Pyrazolo[1,5-a]-1,3,5-triazin-4-one (VIIa). A solution of 1.0 g of 5-amino-2-pyrazolecarboxamide and of 3.0 ml of trimethyl orthoformate in 50 ml of CH3CN is refluxed for 36 hours. The mixture is allowed to return to ambient temperature. It is left to crystallize for 2 days. The crystals are filtered off. Recrystallization from CH3CN is carried out. The title product is obtained in the form of colorless crystals.

2-Methylpyrazolo[1,5-a]-1,3,5-triazin-4-one (VIIb). 125 mg of Na are added to 10 ml of anhydrous EtOH. When the Na has been entirely consumed, 200 mg of N-(pyrazol-3-yl)acetamidine acetate.NaCl (VIa) and 605 μl of diethyl carbonate are added to this solution under an inert atmosphere. The mixture is refluxed for 5 hours. It is evaporated to dryness. The product is taken up in 10 ml of ice-cold water. A 0.1N HCl solution is added to pH=7 (controlled with pH paper). The mixture is evaporated to dryness. The product is taken up in 7 ml of ice-cold water. It is left to crystallize for 2 hours. The crystals are filtered off and recrystallization from EtOH/Et2O is carried out. 110 mg of the title product are obtained in the form of colorless crystals. M: 150.14. Yield: 89%. Mp: 268° C. 1H-NMR (300 MHz, CDCl3): 2.32 (s, 3H, CH₃), 6.38 (d, J=1.8, H8 pyrazole), 8.01 (d, J=1.8, H7 pyrazole), 12.48 (broad s, 1H exchangeable, NH).

2-Thioxo-1,2,3,4-tetrahydropyrazolo[1,5-a]-1,3,5-triazin-4-one (VIIc). 676 mg of Na are added, in small fractions, to 20 ml of absolute EtOH. When the Na has been completely consumed, 900 mg of N-ethoxycarbonyl-N′-(pyrazol-3-yl)thiourea are added. The mixture is stirred at ambient temperature for 20 minutes. It is evaporated to dryness. 10 ml of ice-cold H2O are added and the mixture is stirred vigorously for 20 minutes at 0° C. The mixture is filtered and washed twice with 5 ml of EtOH and then twice with 10 ml of Et2O. 671 mg of title product are obtained in the form of a white powder. Yield: 95%. Mp: 295° C. ¹H-NMR (200 MHz, DMSO-d6+1 drop of D2O): 5.51 (d, J=1.5, H8 pyrazole), 7.48 (d, J=1.5, H7 pyrazole).

2-Thiomethylpyrazolo[1,5-a]-1,3,5-triazin-4-one (VIId). 222 μl of MeI are added dropwise to a solution of 600 mg of 2-thioxo-1,2,3,4-tetrahydropyrazolo[1,5-a]-1,3,5-triazin-4-one (VIIc) in 20 ml of EtOH, 3 ml of H₂O and 3 ml of sodium lye. The mixture is stirred at ambient temperature for 20 minutes. The white crystals of the title product (Na salt) are filtered off. The crystals are taken up in 10 ml of H2O and the pH is adjusted to 8 (controlled with pH paper). The product is filtered and washed twice with 2 ml of H2O. 429 mg of the title product are obtained in the form of a white powder. M: 182.21. Yield: 66%. Mp: 257° C. 1H-NMR (200 MHz, 1 drop of DMSO-d6+CDCl₃): 2.25 (s, 3H, CH3), 5.92 (d, J=2.0, 1H, H8 pyrazole), 7.53 (d, J=2.0, 1H, H7 pyrazole).

4-(N-Methyl-N-phenylamino)pyrazolo[1,5-a]-1,3,5-triazine (IXa). A mixture of 1.0 g of pyrazolo[1,5-a]-1,3,5-triazin-4-one (VIIa) in 3 ml of dimethylaniline and 8 ml of POCl3 is refluxed for 2 hours. The POCl₃ is evaporated off. The product is vacuum dried (1 hour). 50 ml of CH2Cl2 are added, along with, dropwise, 3 ml of methylaniline and 6 ml of triethylamine. After 1 hour at ambient temperature, the mixture is evaporated to dryness and 30 ml of ice-cold water are added. The mixture is extracted twice with 30 ml of Et2O, and the organic fractions are dried over Na2SO4 and evaporated to dryness. Purification is carried out by chromatography on silica (1 EtOAc/2 hexane and then 1 EtOAc/1 hexane). The product is recrystallized from hexane. Yield: 88%. 1H-NMR (300 MHz, CDCl3): 4.10 (s, 3H, CH3), 6.64 (d, 1H, 1H pyrazole), 7.44-7.72 (m, 5H, 5H Ar), 8.03 (d, 1H, 1H pyrazole), 8.48 (s, 1H, 2-H).

2-Methyl-4-(N-methyl-N-phenylamino)pyrazolo[1,5-a]-1,3,5-triazine (IXb). By replacing, in example IXa, the pyrazolo[1,5-a]-1,3,5-triazin-4-one (VIIa) with 2-methylpyrazolo[1,5-a]-1,3,5-triazin-4-one (VIIb), the title product is obtained in the same way (yield: 92%). Mp: 116° C.

2-Methyl-4-[N-methyl-N-(4-nitrophenyl)amino]-8-nitropyrazolo[1,5-a]-1,3,5-triazine (Xa). 2.3 g of 2-methyl-4-(N-methyl-N-phenylamino)pyrazolo[1,5-a]-1,3,5-triazine (IXb) are added to 18 ml of fuming HNO3 at 0° C. The reaction medium turns a dark red color. After 10 minutes at 0° C., 300 ml of an H2O/ice mixture are added. A green precipitate forms. It is filtered off and washed twice with 20 ml of H2O, twice with 6 ml of MeOH and twice with 10 ml of Et2O. Purification is carried out by chromatography (50 CH2Cl2/50 Et2O). The product is triturated in 15 ml of Et2O. Filtration is carried out, followed by washing with 2 ml of Et2O. 2.7 g of the title product are obtained in the form of a cream powder (yield: 85%). Mp: 256° C. 1H-NMR (300 MHz, CDCl3): 2.74 (s, 3H, CH3), 3.83 (s, 3H, NCH3), 7.85 (AB system, Δd=0.94, JAB=8.7, 4H, NO2Ph), 8.28 (s, H7 pyrazole).

8-Amino-4-[N-(4-aminophenyl)-N-methylamino]-2-methylpyrazolo[1,5-a]-1,3,5-triazine (XIa). A solution/suspension of 60 mg of 2-methyl-4-[N-methyl-N-(4-nitrophenyl)amino]-8-nitropyrazolo[1,5-a]-1,3,5-triazine (Xa), and 60 mg of palladium-on-charcoal in 30 ml of MeOH is hydrogenated at atmospheric pressure for 2 hours. It is filtered through celite. Washing is carried out twice with 10 ml of MeOH. The product is evaporated to dryness. Purification is carried out by chromatography (50 CH2Cl2/10 EtOH/40 EtOAc) then (40 CH2Cl2/20 EtOH/40 EtOAc). A yellow oil is obtained, which crystallizes when it is triturated in a minimum amount of Et2O (yield: 68%). Mp: 166° C. 1H-NMR (200 MHz, CDCl3): 2.54 (s, 3H, CH3), 3.66 (s, 3H, NCH3) 6.83 (AB system, Δd=0.29, J=8.6, 4H, NH2Ph), 7.50 (s, H7 pyrazole).

8-Acetamido-4-[N-(4-acetamidophenyl)-N-methylamino]-2-methylpyrazolo[1,5-a]-1,3,5-triazine (XIIa). 47 μl of acetyl chloride are added, dropwise at 0° C., to a solution of 80 mg of 8-amino-4-[N-(4-aminophenyl)-N-methylamino]-2-methylpyrazolo[1,5-a]-1,3,5-triazine (XIa) in 7 ml of anhydrous CH2Cl2. 96 μl of triethylamine are added dropwise. The mixture is allowed to return to ambient temperature. It is evaporated to dryness. 15 ml of H2O are added and the mixture is extracted 3 times with 10 ml of CH2Cl2. Drying is carried out over Na2SO4. The product is evaporated to dryness. Purification is carried out by chromatography (50 CH2Cl2/40 EtOAc/10 EtOH) then (40 CH2Cl2/40 EtOAc/20 EtOH). The product is evaporated to dryness. It is triturated in 10 ml of Et2O. 88 mg of the title product are obtained in the form of a white powder (yield: 84%). Mp: 1580C. 1H-NMR (200 MHz, CDCl3): 2.21 (s, 6H, 2×CH3CO), 2.56 (s, 3H, CH3), 3.74 (s, 3H, NCH3), 7.53 (AB system, Δd=0.41, JAB=8.8, 4H, CONHPh), 7.60 (broad s, 2H, 2 exchangeable NH), 8.35 (s, H7 pyrazole).

8-Iodo-4-(N-methyl-N-phenylamino)pyrazolo[1,5-a]-1,3,5-triazine (XIIIa). 140 mg of NIS are added to a solution of 100 mg of 4-(N-methyl-N-phenylamino)pyrazolo[1,5-a]-1,3,5-triazine (IXa) in 10 ml of CHCl3. The mixture is refluxed for 30 minutes. It is evaporated to dryness. Purification is carried out by chromatography (EtOAc/hexane, 1:3). The product is recrystallized from EtOH. The title product is obtained in the form of colorless crystals. Yield: 91%. Mp: 193° C. 1H-NMR (300 MHz, CDCl3): 3.82 (s, 3H, NCH3), 7.19-7.44 (m, 5H, Ph), 7.77 (s, 1H, H7 pyrazole), 8.3 (s, 1H, H2 pyrazole).

8-Iodo-2-methyl-4-(N-methyl-N-phenylamino)pyrazolo-[1,5-a]-1,3,5-triazine (XIIIb). By replacing, in example XIIIa, the 4-(N-methyl-N-phenylamino)pyrazolo[1,5-a]-1,3,5-triazine (IXa) with 2-methyl-4-(N-methyl-N-phenylamino)pyrazolo[1,5-a]-1,3,5-triazine (IXb), the title product is obtained in the same way (yield: 78%).

### EXAMPLE 2

### Synthesis of the Compounds of the Formula Ib

Methyl 4-[(hydroxy)[4-(N-methyl-N-phenylamino)pyrazolo[1,5-a]-1,3,5-triazin-8-yl]methyl]benzoate (Ib1). 220 μl of n-BuLi at 15% in hexane are added, at −78° C. and under argon, to a solution of 160 mg of 8-iodo-4-(N-methyl-N-phenylamino)pyrazolo[1,5-a]-1,3,5-triazine (XIIIa) in 25 ml of anhydrous THF. After 5 minutes at −78° C., 115 mg of methyl 4-formylbenzoate are added. The mixture is allowed to return to ambient temperature. It is evaporated to dryness. 30 ml of H2O are added and the mixture is extracted 3 times with 30 ml of CH2Cl2. Drying is carried out over Na2SO4, followed by filtration. The product is evaporated to dryness. Purification is carried out by chromatography (1 EtOAc/1 hexane). The product is recrystallized from Et2O/hexane. The title product is obtained in the form of colorless crystals (yield=93%). Mp: 68° C. 1H-NMR (300 MHz, CDCl3) 3.80 (s, 3H, CH3), 3.91 (s, 3H, CH3), 6.22 (s, 1H, CH), 7.17-7.55 (m, 8H, 8 ArH), 8.01 (d, J=8.2, 2H, 2 CH), 8.21 (s, 1H, 1 ArH).

8-[(2-Chlorophenyl)(hydroxy)methyl]-4-(N-methyl-N-phenylamino)-2-n-propylpyrazolo[1,5-a]-1,3,5-triazine (Ib2). 640 μl (1.52 mmol, 1.2 eq) of n-butyllithium (2.37 M in heptane) are added, under an inert atmosphere and at −78° C., to a solution of 8-iodo-4-(N-methyl-N-phenylamino)-2-n-propylpyrazolo[1,5-a]-1,3,5-triazine (500 mg, 1.27 mmol) in 30 ml of THF. The reaction mixture is stirred at −78° for 5 min. A solution of 2-chlorobenzaldehyde (0.17 ml, 1.52 mmol, 1.2 eq) in 5 ml of THF is then added dropwise and the reaction medium is stirred at −78° C. for a further 1 h, and is then hydrolyzed by means of the addition of water, and concentrated under reduced pressure. The oily residue obtained is divided between ethyl acetate and water. The organic phase is washed with a saturated sodium chloride solution, dried (Na2SO4) and evaporated. The residue is purified by flash chromatography on silica gel (eluent: petroleum ether/EtOAc, 8:2) so as to give the title product (383 mg, 74%) in the form of a colorless solid: Mp=153-155° C. (methanol); 1H-NMR (300 MHz, CDCl3) d 1.01 (t, 3H, J=7.3 Hz, CH3), 1.78-1.91 (m, 2H, CH2), 2.73 (t, 2H, J=7.3 Hz, CH2), 3.71 (s, 3H, CH3), 4.63 (d, 1H, J=4.3 Hz, OH), 6.49 (d, 1H, J=4.3 Hz, CH), 7.13-7.20 (m, 3H, H Ar), 7.28-7.38 (m, 6H, H Ar), 7.74-7.78 (m, 1H, H Ar); 13C-NMR (75 MHz, CDCl3) δ 14.0 (CH3), 21.2 (CH2), 40.6 (CH2), 42.1 (CH3), 65.0 (CH), 109.4 (C), 126.3 (2 CH), 127.1 (CH), 127.2 (CH), 127.8 (CH), 128.5 (CH), 129.0 (2 CH), 129.3 (CH), 132.0 (C), 141.2 (C), 143.6 (CH), 144.6 (C), 148.6 (C), 149.1 (C), 165.6 (C); MS (SI) m/z 390 (M++1, 35Cl), 392 (M++1, 37Cl).

8-(2-Chlorobenzoyl)-4-(N-methyl-N-phenylamino)-2-n-propylpyrazolo[1,5-a]-1,3,5-triazine (Ib3). 700 mg (8.05 mmol, 8 eq) of manganese dioxide are added, under an inert atmosphere, to a solution of 8-[(2-chlorophenyl)(hydroxy)methyl]-4-(N-methyl-N-phenylamino)-2-n-propylpyrazolo[1,5-a]-1,3,5-triazine (Ib2) (380 mg, 0.93 mmol) in 20 ml of CH2Cl2. The mixture is stirred overnight at ambient temperature, and then filtered through celite and evaporated. The residue is purified by column chromatography on silica gel (petroleum ether/EtOAc: 8/2) so as to give the title compound (346 mg, 90%) in the form of a colorless solid: Mp=149-151° C. (methanol); 1H-NMR (300 MHz, CDCl3) δ 0.93 (t, 3H, J=7.3 Hz, CH3), 1.65-1.73 (m, 2H, CH2), 2.73 (t, 2H, J=7.3 Hz, CH2), 3.74 (s, 3H, CH3), 7.15-7.19 (m, 2H, H Ar), 7.29-7.40 (m, 7H, H Ar), 7.95 (s, 1H, H Ar); 13C-NMR (75 MHz, CDCl3) δ 13.9 (CH3), 20.4 (CH2), 40.8 (CH2), 42.3 (CH3), 109.1 (C), 126.2 (2 CH), 126.5 (CH), 127.5 (CH), 129.1 (2 CH), 129.6 (CH), 130.5 (CH), 130.9 (C), 132.0 (C), 140.2 (C), 144.3 (C), 146.9 (CH), 149.2 (C), 152.3 (C), 170.2 (C), 187.3 (CO); MS (SI) m/z 406 (M++1, 35Cl), 408 (M++1, 37C1).

8-(2-Chlorobenzoyl)-4-(N-methylamino)-2-n-propylpyrazolo[1,5-a]-1,3,5-triazine (Ib4). A solution of 8-(2-chlorobenzoyl)-4-(N-methyl-N-phenylamino)-2-n-propylpyrazolo[1,5-a]-1,3,5-triazine (Ib3) (320 mg, 0.79 mmol) and of methylamine (33 wt % in ethanol, 0.2 ml, 1.6 mmol, 2 eq) in 10 ml of ethanol is stirred in a sealed tube overnight at 70° C. After cooling, the ethanol is evaporated off. The residue is purified by column chromatography on silica gel (eluent: CH2Cl2/EtOAc, 9.5/0.5) so as to give the title compound (172 mg, 66%) in the form of a colorless solid: Mp=116-118° C. (methanol). 1H-NMR (300 MHz, CDCl3) δ 0.91 (t, 3H, J=7.3 Hz, CH3), 1.62-1.72 (m, 2H, CH2), 2.71 (t, 2H, J=7.3 Hz, CH2), 3.24 (d, 3H, J=5.1 Hz, CH3), 6.54 (broad s, 1H, NH), 7.31-7.45 (m, 4H, H Ar), 8.26 (s, 1H, H Ar); 13C-NMR (75 MHz, CDCl3) δ 13.9 (CH3), 20.6 (CH2), 27.4 (CH2), 41.1 (CH3), 110.6 (C), 126.7 (CH), 128.8 (CH), 129.8 (CH), 130.7 (CH), 131.1 (CH), 140.1 (C), 147.6 (CH), 149.4 (C), 149.7 (C), 171.3 (C), 187.5 (CO); MS (SI) m/z 330 (M++1, 35C1), 332 (M++1, 37C1); HRMS (IC) for C16H17ClN5O; calculated: 330.1121; found: 330.1123.

Ethyl 3-[4-(N-methyl-N-phenylamino)pyrazolo[1,5-a]-1,3,5-triazin-8-yl]acrylate (Ib5). A mixture of 1.0 g of 8-iodo-4-(N-methyl-N-phenylamino)pyrazolo[1,5-a]-1,3,5-triazine (XIIIa), 2.5 ml of methyl acrylate, 450 mg of PdCl2(dppf) and 2.0 g of tetrabutylammonium iodide in a mixture of DMF:H2O:TEA (25:5:5) is heated at 55° C. for 3 hours under an inert atmosphere. The reaction medium is evaporated to dryness. The residue is taken up in 200 ml of EtOAc and washed twice with 100 ml of H2O. The organic fractions are dried over Na2SO4. The product is evaporated to dryness. The residue is purified by chromatography on silica (EtOAc/hexane, 1:3). The product is recrystallized from Et2O/hexane. 790 mg of title product are obtained in the form of colorless crystals. Mp: 139° C. 1H-NMR (75 MHz, CDCl3): 1.32 (t, J=7.1 Hz, 3H, CH3), 3.82 (s, 3H, NCH3), 4.24 (m, J=7.1 Hz, 2H, CH2), 6.63 (d, J=15.9 Hz, 1H, CH), 7.20-7.46 (m, 5H, 5 ArH), 7.78 (d, J=15.9 Hz, 1H, CH), 7.90 (s, 1H, CH), 8.31 (s, 1H, CH). 13C-NMR (300 MHz, CDCl3): 16.0, 44.1, 61.7, 107.3, 118.3, 127.8, 129.2, 130.8, 134.6, 145.8, 151.0, 151.6, 155.6, 169.0.

Ethyl 3-[4-(N-methyl-N-phenylamino)pyrazolo[1,5-a]-1,3,5-triazin-8-yl]propionate (Ib6). A suspension of 1.2 g of ethyl 3-[4-(N-methyl-N-phenylamino)pyrazolo[1,5-a]-1,3,5-triazin-8-yl]acrylate (Ib5) and of 500 mg of Pd/C (10%) in 80 ml of methanol is hydrogenated at ambient temperature and at atmospheric pressure for 6 hours. The reaction medium is filtered through filter paper. Recrystallization from Et2O/hexane is carried out. 1.1 g of the title product are obtained in the form of colorless crystals. Mp=74° C. 1H-NMR (75 MHz, CDCl3): 1.27 (t, J=7.2 Hz, 3H, CH3), 2.66 (t, J=7.4 Hz, 2H, CH2), 2.99 (t, J=7.4 Hz, 2H, CH2), 3.80 (s, 3H, NCH3), 4.11 (m, J=7.2 Hz, 2H, CH2), 7.17-7.41 (m, 5H, 5 ArH), 7.68 (s, 1H, CH), 8.19 (s, 1H, CH). 13C-NMR (300 MHz, CDCl3): 15.8, 19.8, 36.2, 43.8, 61.9, 109.1, 127.7, 128.8, 130.6, 146.2, 149.6, 151.6, 153.2, 174.4.

3-[4-(N-Methyl-N-phenylamino)pyrazolo[1,5-a]-1,3,5-triazin-8-yl]propionic acid (Ib7). An equimolar solution of ethyl 3-[4-(N-methyl-N-phenylamino)pyrazolo[1,5-a]-1,3,5-triazin-8-yl]propionate Ib6 and of NaOH in a 1:9 H2O/EtOH mixture is stirred at ambient temperature for 24 hours. The precipitate is filtered off and taken up in a minimum of water, and the pH is then brought to 3-4 using 1N HCl. The precipitate is filtered off. The title product is obtained in the form of colorless crystals. 1H-NMR (300 MHz, CDCl3): 2.73 (t, J=7.1 Hz, 2H, CH2), 3.01, (t, J=7.1 Hz, 2H, CH2), 3.80 (s, 3H, CH3), 7.18-7.41 (m, 5H, 5 ArH), 7.69 (s, 1H, 1 ArH), 8.20 (s, 1H, 1 ArH)

Methyl 4-[[1-oxo-3-[4-(N-methyl-N-phenylamino)pyrazolo[1,5-a]-1,3,5-triazin-8-yl]propyl]amino]benzoate (Ib8). A solution of 380 mg of O-benzotriazol-1-yl-N,N,N′,N′-tetramethyluronium (HBTU), 400 μl of N-methylmorpholine and 297 mg of 3-[4-(N-methyl-N-phenylamino)pyrazolo[1,5-a]-1,3,5-triazin-8-yl]propionic acid (Ib7) in 4 ml of anhydrous DMF is stirred at ambient temperature for one hour. 152 mg of methyl 4-aminobenzoate are added and the reaction medium is stirred for 48 hours. It is then diluted with 100 ml of EtOAc and washed twice with 20 ml of water. The organic fractions are dried (Na2SO4). The product is evaporated to dryness. Purification is carried out by chromatography on silica (EtOAc/hexane, 1:1 then EtOAc). The product is recrystallized from EtOH/Et2O. The title product is obtained in the form of a white powder (yield=78%). 1H-NMR (300 MHz, CDCl3): 2.83 (t, J=7.0 Hz, 2H, CH2), 3.12 (t, J=7.0 Hz, 2H, CH2), 3.83 (s, 3H, CH3), 3.91 (s, 3H, CH3), 7.18-7.21 (m, 2H, 2 ArH), 7.37-7.46 (m, 3H, 3 ArH), 7.59 (d, J=8.5 Hz, 2H, 2 CH), 7.71 (s, 1H, 1 ArH), 7.99 (d, J=8.5 Hz, 2H, 2 CH), 8.17 (s1, 1H, NH), 8.23 (s, 1H, 1 ArH).

8-Benzoyl-2-methyl-4-(N-methyl-N-phenylamino)pyrazolo[1,5-a]-1,3,5-triazine (Ib9). 580 μl of benzoyl chloride are added, under argon, to 227 mg of 2-methyl-4-(N-methyl-N-phenylamino)pyrazolo[1,5-a]-1,3,5-tri-azine (IXb). 588 μl of SnCl4 are added dropwise. The reaction medium is heated at 60° C. for 12 hours. The reaction medium turns black. It is poured into 40 ml of H2O and extracted 3 times with 40 ml of EtOAc. Drying is carried out over Na2SO4, followed by filtration. The product is evaporated to dryness. Purification is carried out by chromatography (1 EtOAc/2 hexane). 292 mg of an oil are obtained, which crystallizes slowly. Yield: 85%. Mp: 121° C. 1H-NMR (200 MHz, CDCl3): 2.68 (s, 3H, CH3), 3.79 (s, 3H, NCH3), 7.20-7.60 (m, 8H Ar), 7.84-7.90 (m, 2H Ar), 8.05 (s, H7 pyrazole).

Ethyl 2-methyl-4-(N-methyl-N-phenylamino)pyrazolo-[1,5-a]-1,3,5-triazine-6-carboxylate (Ib10). The benzoyl chloride, in example Ib9, is replaced with oxalyl chloride and, at the end of the reaction, the product is evaporated to dryness. 20 ml of absolute EtOH are added and the reaction medium is refluxed for 4 hours. It is evaporated to dryness. 40 ml of an H2O/ice mixture are added. The reaction medium is extracted 3 times with 30 ml of EtOAc. Drying is carried out over Na2SO4. Partial purification is carried out by chromatography (1 EtOAc/1 Hex). The product is recrystallized from EtOH. Mp: 202° C. MS (FAB, M+H+): 312. 1H-NMR (300 MHz, DMSO-d6): 1.36 (t, J=7.1, 3H, CH2CH3), 2.66 (s, 3H, CH3), 3.74 (s, 3H, NCH3), 4.36 (q, J=7.1, 2H, CH2CH3), 7.14-7.18 (m, 2H Ar), 7.35-7.41 (m, 3H Ar), 8.06 (s, H7 pyrazole).

tert-Butyl 3-[4-(N-methyl-N-phenylamino)pyrazolo-[1,5-a]-1,3,5-triazin-8-yl]acrylate (Ib11). By replacing, in example Ib5, the ethyl acrylate with tert-butyl acrylate, the title product (87%) is obtained, in the same way, in the form of a colorless solid.

tert-Butyl 3-[4-(N-methyl-N-phenylamino)pyrazolo-[1,5-a]-1,3,5-triazin-8-yl]propionate (Ib12). By replacing, in example Ib6, the ethyl 3-[4-(N-methyl-N-phenylamino)pyrazolo[1,5-a]-1,3,5-triazin-8-yl]acrylate (Ib5) with tert-butyl 3-[4-(N-methyl-N-phenylamino)pyrazolo[1,5-a]-1,3,5-triazin-8-yl]acrylate (Ib11), the title product (76%) is obtained, in the same way, in the form of a colorless solid. This product can be converted to 3-[4-(N-methyl-N-phenylamino)pyrazolo[1,5-a]-1,3,5-triazin-8-yl]propionic acid (Ib7) by simple cleavage of the tert-butyl ester using trifluoroacetic acid in dichloromethane (yield: 95%).

4-(N-Methyl-N-phenylamino)-8-phenylpyrazolo[1,5-a]-1,3,5-triazine (Ib13). 80 mg of 8-iodo-4-(N-methyl-N-phenylamino)pyrazolo[1,5-a]-1,3,5-triazine (XIIIb) are dissolved in 6 ml of degassed toluene. 25 mg of tetrakistriphenylphosphine palladium(0); 210 μl of 2M Na2CO3 in H2O and 30 mg of benzeneboronic acid dissolved in 30 μl of EtOH are added. The reaction medium is heated at 90° C. for 15 hours under argon. It is evaporated to dryness. Purification is carried out by chromatography (50 EtOAc/50 hexane). 40 mg of the title product are obtained in the form of a cream powder. Yield: 78%.

4-(N-Methyl-N-phenylamino)-8-(4-fluorophenyl)pyrazolo[1,5-a]-1,3,5-triazine (Ib14). By replacing, in example Ib13, the benzeneboronic acid with 4-fluorobenzeneboronic acid, the title product (78%) is obtained, in the same way, in the form of a colorless solid.

8-[(3-Furyl)(hydroxy)methyl]-4-(N-methyl-N-phenylamino)-2-n-propylpyrazolo[1,5-a]-1,3,5-triazine (Ib15). By replacing, in example Ib2, the 2-chlorobenzaldehyde with 3-furaldehyde, the title product (65%) is obtained, in the same way, in the form of a colorless solid. Mp=142-144° C. (methanol); 1H-NMR (300 MHz, CDCl3) δ 1.02 (t, 3H, J=7.3 Hz, CH3), 1.79-1.92 (m, 2H, CH2), 2.75 (t, 2H, J=7.3 Hz, CH2), 3.70 (d, 1H, J=4.5 Hz, OH), 3.73 (s, 3H, CH3), 6.08 (d, 1H, J=4.5 Hz, CH), 6.43 (broad s, 1H, H Ar), 7.15-7.18 (m, 2H, H Ar), 7.32-7.41 (m, 5H, H Ar), 7.54 (m, 1H, H Ar); 13C-NMR (75 MHz, CDCl3) δ 14.0 (CH3), 21.2 (CH2), 40.6 (CH2), 42.0 (CH3), 60.8 (CH), 109.4 (CH), 110.3 (C), 126.2 (2 CH), 127.1 (CH), 128.4 (C), 129.0 (2 CH), 139.5 (CH), 143.2 (CH), 143.4 (CH), 144.6 (C), 148.3 (C), 149.1 (C), 165.5 (C); MS (SI) m/z 364 (M++1).

8-(3-Furylmethyl)-2-n-propyl-4-(N-methyl-N-phenylamino)pyrazolo[1,5-a]-1,3,5-triazine (Ib16). 312 mg (8.25 mmol, 9 eq) of sodium borohydride are added, at 0° C. and under an inert atmosphere, to 2 ml of trifluoroacetic acid. A solution of 8-[(3-furyl)(hydroxy)methyl]-4-(N-methyl-N-phenylamino)-2-n-propylpyrazolo[1,5-a]-1,3,5-triazine (Ib15) (333 mg, 0.92 mmol) in dichloromethane (5 ml) is added dropwise to this mixture at 15° C. The solution is then stirred at ambient temperature for 2 h, and then diluted by adding water and basified by adding sodium hydroxide. The product is extracted with dichloromethane, dried (MgSO4) and evaporated under reduced pressure. The residue is purified by column chromatography on silica gel (eluent: petroleum ether/EtOAc: 8/2) so as to give the title compound (286 mg, 90%) in the form of a colorless oil. 1H-NMR (300 MHz, CDCl3) δ 1.03 (t, 3H, J=7.3 Hz, CH3), 1.81-1.94 (m, 2H, CH2), 2.77 (t, 2H, J=7.3 Hz, CH2), 3.73 (s, 3H, CH3), 3.80 (s, 2H, CH2), 6.31 (broad s, 1H, H Ar), 7.15-7.18 (m, 2H, H Ar), 7.22 (broad s, 1H, H Ar), 7.30-7.40 (m, 4H, H Ar), 7.56 (s, 1H, H Ar); 13C-NMR (75 MHz, CDCl3) δ 14.1 (CH3), 18.4 (CH2), 21.4 (CH2), 41.0 (CH2), 42.0 (CH2), 106.5 (C), 111.4 (CH), 124.1 (C), 126.1 (2 CH), 126.8 (CH), 128.9 (2 CH), 139.4 (CH), 142.9 (CH), 144.9 (CH), 145.0 (C), 148.5 (C), 149.4 (C), 164.9 (C); MS (SI) m/z 348 M++1).

8-(3-Furylmethyl)-2-n-propyl-4-(N-methylamino)pyrazolo[1,5-a]-1,3,5-triazine (Ib17). By replacing, in example Ib4, the 8-(2-chlorobenzoyl)-4-(N-methyl-N-phenylamino)-2-n-propylpyrazolo[1,5-a]-1,3,5-triazine (Ib3) with 8-(3-furylmethyl)-2-n-propyl-4-(N-methyl-N-phenylamino)pyrazolo[1,5-a]-1,3,5-triazine (Ib16), the title product (90%) is obtained, in the same way, in the form of a colorless oil. 1H-NMR (300 MHz, CDCl3) δ 1.02 (t, 3H, J=7.3 Hz, CH3), 1.80-1.93 (m, 2H, CH2), 2.74 (t, 2H, J=7.3 Hz, CH2), 3.20 (d, 3H, J=5.1 Hz, CH3), 3.84 (s, 2H, CH2), 6.34 (broad s, 1H, H Ar), 6.49 (broad s, 1H, NH), 7.26 (broad s, 1H, H Ar), 7.34 (broad s, 1H, H Ar), 7.74 (s, 1H, H Ar); 13C-NMR (75 MHz, CDCl3) δ 14.1 (CH3), 18.4 (CH2), 21.7 (CH2), 27.3 (CH3), 41.4 (CH2), 107.8 (C)—, 111.4 (CH), 124.2 (C), 139.5 (CH), 143.0 (CH), 145.0 (CH), 146.3 (C), 149.4 (C), 166.2 (C); MS (SI) m/z 272 (M++1); HRMS (IC) for C14H18N50; calculated: 272.1511; found: 272.1513.

8-[(Hydroxy)(2-thienyl)methyl]-4-(N-methyl-N-phenylamino)-2-n-propylpyrazolo[1,5-a]-1,3,5-triazine (Ib18). By replacing, in example Ib2, the 2-chlorobenzaldehyde with 2-thiophenecarboxaldehyde, the title product (68%) is obtained, in the same way, in the form of a colorless solid: Mp=150-152° C. (methanol). 1H-NMR (300 MHz, CDCl3) δ 1.02 (t, 3H, J=7.3 Hz, CH3), 1.79-1.91 (m, 2H, CH2), 2.75 (t, 2H, J=7.3 Hz, CH2), 3.74 (s, 3H, CH3), 4.15 (d, 1H, J=4.5 Hz, OH), 6.35 (d, 1 h, J=4.5 Hz, CH), 6.91-6.97 (m, 2H, H Ar), 7.15-7.19 (m, 2H, H Ar), 7.23 (dd, 1H, J=1.3, 4.9 Hz, H Ar), 7.32-7.40 (m, 3H, H Ar), 7.56 (s, 1H, H Ar); 13C-NMR (75 MHz, CDCl3) d 14.0 (CH3), 21.2 (CH2), 40.6 (CH2), 42.1 (CH3), 64.5 (CH), 110.3 (C), 124.3 (CH), 124.9 (CH), 126.3 (CH), 126.6 (CH), 127.2 (CH), 129.0 (2 CH), 143.4 (CH), 144.6 (C), 148.0 (C), 148.4 (C), 149.1 (C), 165.7 (C); MS (SI) m/z 380 (M++1).

4-(N-Methyl-N-phenylamino)-2-n-propyl-8-(2-thienylmethyl)pyrazolo[1,5-a]-1,3,5-triazine (Ib19). By replacing, in example Ib16, the 8-[(3-furyl)(hydroxy)methyl]-4-(N-methyl-N-phenylamino)-2-n-propylpyrazolo[1,5-a]-1,3,5-triazine (Ib15) with 8-[(hydroxy)(2-thienyl)methyl]-4-(N-methyl-N-phenylamino)-2-n-propylpyrazolo[1,5-a]-1,3,5-triazine (Ib18), the title product (90%) is obtained, in the same way, in the form of a colorless oil: 1H-NMR (300 MHz, CDCl3) δ 1.03 (t, 3H, J=7.3 Hz, CH3), 1.81-1.93 (m, 2H, CH2), 2.77 (t, 2H, J=7.3 Hz, CH2), 3.73 (s, 3H, CH3), 4.21 (s, 2H, CH2), 6.83-6.89 (m, 2H, H Ar), 7.09 (dd, 1H, J=1.1, 5.1 Hz, H Ar), 7.15-7.18 (m, 2H, H Ar), 7.28-7.40 (m, 3H, H Ar), 7.60 (s, 1H, H Ar); 13C-NMR (75 MHz, CDCl3) δ 14.1 (CH3), 21.4 (CH2), 23.1 (CH2), 40.9 (CH2), 42.0 (CH3), 106.6 (C), 123.5 (CH), 124.8 (CH), 126.1 (2 CH), 126.8 (CH), 126.9 (CH), 128.9 (2 CH), 143.9 (C), 144.9 (CH), 145.0 (C), 148.5 (C), 149.3 (C), 165.1 (C); MS (SI) m/z 364 (M++1).

4-(N-methyl-N-phenylamino)-2-n-propyl-8-(2-thienylmethyl)pyrazolo[1,5-a]-1,3,5-triazine (Ib19). By replacing, in example Ib4, the 8-(2-chlorobenzoyl)-4-(N-methyl-N-phenylamino)-2-n-propylpyrazolo[1,5-a]-1,3,5-triazine (Ib3) with 4-(N-methyl-N-phenylamino)-2-n-propyl-8-(2-thienylmethyl)pyrazolo[1,5-a]-1,3,5-triazine (Ib19), the title product (92%) is obtained, in the same way, in the form of a colorless solid. Mp=53-55° C.; 1H-NMR (300 MHz, CDCl3) δ 1.02 (t, 3H, J=7.3 Hz, CH3), 1.81-1.93 (m, 2H, CH2), 2.75 (t, 2H, J=7.3 Hz, CH2), 3.20 (d, 3H, J=5.1 Hz, CH3), 4.25 (s, 2H, CH2), 6.57 (broad s, 1H, NH), 6.87-6.92 (m, 2H, HAr), 7.11 (dd, 1H, J=1.1, 5.1 Hz, HAr), 7.80 (s, 1H, HAr); 13C-NMR (75 MHz, CDCl3) δ 14.1 (CH3), 21.7 (CH2), 23.2 (CH2), 27.3 (CH3), 41.4 (CH2), 107.9 (C), 123.7 (CH), 124.9 (CH), 126.9 (CH), 144.0 (C), 145.1 (CH), 146.3 (C), 149.4 (C), 166.4 (C); MS (SI) m/z 288 (M++1).

4-(N-Cyclopropylamino)-2-n-propyl-8-[(2-thienyl)methyl]pyrazolo[1,5-a]-1,3,5-triazine (Ib21). By replacing, in example Ib4,8-(2-chlorobenzoyl)-4-(N-methyl-N-phenylamino)-2-n-propylpyrazolo[1,5-a]-1,3,5-triazine (Ib3) with 4-(N-methyl-N-phenylamino)-2-n-propyl-8-[(2-thienylmethyl]pyrazolo[1,5-a]-1,3,5-triazine (Ib19) and the methylamine with cyclopropylamine, the title product (80%) is obtained, in the same way, in the form of a colorless solid. Mp=52-53° C.; 1H-NMR (300 MHz, CDCl3) δ 0.71-0.76 (m, 2H, CH2), 0.91-0.98 (m, 2H, CH2), 1.03 (t, 3H, J=7.3 Hz, CH3), 1.82-1.94 (m, 2H, CH2), 2.78 (t, 2H, J=7.3 Hz, CH2), 2.99-3.07 (m, 1H, CH), 4.24 (s, 2H, CH2), 6.55 (broad s, 1H, NH), 6.86-6.92 (m, 2H, HAr), 7.11 (dd, 1H, J=1.1, 5.1 Hz, HAr), 7.83 (s, 1H, HAr); 13C-NMR (75 MHz, CDCl3) δ 7.2 (2 CH2), 14.1 (CH3), 21.7 (CH2), 23.2 (CH2), 23.4 (CH), 41.4 (CH2), 108.0 (C), 123.7 (CH), 124.9 (CH), 126.9 (CH), 144.0 (C), 145.0 (CH), 146.2 (C), 149.7 (C), 166.5 (C); MS (EI) m/z 313 (M).

N-[2-(3,4-Dihydroxyphenyl)ethyl]-3-[4-(N-methyl-N-phenylamino)pyrazolo[1,5-a]-1,3,5-triazin-8-yl]propionamide (Ib22). By replacing, in example Ib8, the methyl 4-aminobenzoate with 2-(3,4-dihydroxyphenyl)ethylamine, the title product (22%) is obtained, in the same way, in the form of a colorless solid: MS (SI) m/z 433 (M++1).

3-[4-(N-Methyl-N-phenylamino)pyrazolo[1,5-a]-1,3,5-triazin-8-yl)-N-[3-(2-oxopyrrolidin-1-yl)propyl]propionamide (Ib23). 41 mg (0.34 mmol) of DMAP are added, at 0° C. and under argon, to a solution of N-(3-aminopropyl)-2-pyrrolidinone (0.071 ml, 0.50 mmol) in dichloromethane (8 ml). 3-[4-(N-Methyl-N-phenylamino)pyrazolo[1,5-a]-1,3,5-triazin-8-yl]propionic acid (Ib7) (100 mg, 0.34 mmol) and then EDCI (78 mg, 0.40 mmol) are successively added to the reaction medium. The final solution is stirred at ambient temperature overnight. After the addition of water and extraction, the organic phase is washed with a saturated sodium chloride solution. After drying and evaporation of the organic phase, the crude product is purified by flash chromatography on silica gel (eluent: CH2Cl2/MeOH, 92:8) so as to give the title product (132 mg, 93%, gum). 1H-NMR (300 MHz, CDCl3) δ 1.58-1.67 (m, 2H, CH2), 1.97-2.07 (m, 2H, CH2), 2.38 (t, 2H, J=7.9 Hz, CH2), 2.54 (t, 2H, J=7.5 Hz, CH2), 3.01 (t, 2H, J=7.5 Hz, CH2), 3.15 (broad q, 2H, J=6.2 Hz, CH2), 3.26 (t, 2H, J=7.2 Hz, CH2), 3.36 (t, 2H, J=7.2 Hz, CH2), 3.79 (s, 3H, CH3), 6.73 (broad t, 1H, J=5.9 Hz, NH), 7.16-7.19 (m, 2H, HAr), 7.30-7.42 (m, 3H, HAr), 7.67 (s, 1H, HAr), 8.16 (s, 1H, HAr); 13C-NMR (75 MHz, CDCl3) δ 17.7 (CH2), 18.8 (CH2), 26.4 (CH2), 30.7 (CH2), 35.6 (CH2), 36.7 (CH2), 39.4 (CH2), 42.1 (CH3), 47.1 (CH2), 107.7 (C), 126.0 (2 CH), 127.0 (CH), 128.9 (2 CH), 144.5 (C), 144.6 (CH), 147.7 (C), 149.9 (C), 151.4 (CH), 172.1 (CO), 175.5 (CO); MS (SI) m/z 422 (M++1).

N-[2-Hydroxy-2-(3,4-dihydroxyphenyl)ethyl]-3-[4-(N-methyl-N-phenylamino)pyrazolo[1,5-a]-1,3,5-triazin-8-yl]propionamide (Ib24). By replacing, in example Ib8, the methyl 4-aminobenzoate with 2-(hydroxy)-2-(3,4-dihydroxyphenyl)ethylamine, the title product (22%) is obtained, in the same way, in the form of a colorless solid: MS (SI) m/z 449 (M++1).

4-(N-Methyl-N-phenylamino)-8-(β-D-glycero-pentofuran-3′-ulos-1′-yl)pyrazolo[1,5-a]-1,3,5-triazine (Ib25). A mixture of 62 mg of bis(dibenzylideneacetone)Pd(0) and of 66 mg of triphenylarsine in 5 ml of anhydrous acetonitrile is stirred for 15 minutes under an inert atmosphere. This complex is transferred, by means of a syringe, into a solution of 500 mg of 8-iodo-4-(N-methyl-N-phenylamino)pyrazolo[1,5-a]-1,3,5-triazine (XIIIa), 200 mg of 1,4-anhydro-2-deoxy-D-erythro-pent-1-enitol and 380 μl of tri-n-butylamine in 15 ml of anhydrous acetonitrile. The reaction medium is heated at 60° C. for 12 hours. It is evaporated to dryness. Purification is carried out by chromatography (50 EtOAc/5b Hex), then EtOAc. The product is recrystallized from EtOAc/Hex. 327 mg of title product are obtained in the form of colorless crystals: MS (SI) m/z 340 (M++1).

4-[N-Methyl-N-(4-nitrophenyl)amino]-8-(O-D-glycero-pentofuran-3′-ulos-1′-yl)pyrazolo[1,5-a]-1,3,5-triazine (Ib26). By replacing, in example Ib25, the 8-iodo-4-(N-methyl-N-phenylamino)pyrazolo[1,5-a]-1,3,5-triazine with 8-iodo-4-[N-methyl-N-(4-nitrophenyl)amino]pyrazolo[1,5-a]-1,3,5-triazine, the title product (yield: 61%) is obtained in the same way.

8-(2′-Deoxy-β-D-ribofuranosyl)-4-(N-methyl-N-phenylamino)pyrazolo[1,5-a]-1,3,5-triazine (Ib27). 500 mg of sodium triacetoxyborohydride are added, under argon, to a solution of 165 mg of 4-(N-methyl-N-phenylamino)-8-(β-D-glycero-pentofuran-3′-ulos-1′-yl)pyrazolo[1,5-a]-1,3,5-triazine (Ib25) in 15 ml of anhydrous CH3CN. After 25 minutes, the reaction medium is evaporated to dryness and purification is carried out by chromatography (EtOAc then 9 EtOAc/1 EtOH). After recrystallization (EtOH/Et2O), 120 mg of title product are obtained in the form of colorless crystals: MS (SI) m/z 342 (M++1).

8-(2′-Deoxy-β-D-xylofuranosyl)-4-(N-methyl-N-phenylamino)pyrazolo[1,5-a]-1,3,5-triazine (Ib28). 3.0 ml of KB[CH(CH3)C2H5]3H (K-selectride®) are added, dropwise, under an inert atmosphere and at −78° C., to a solution of 550 mg of 4-(N-methyl-N-phenylamino)-8-(β-D-glycero-pentofuran-3′-ulos-1′-yl)pyrazolo[1,5-a]-1,3,5-triazine (Ib25) in 100 ml of anhydrous THF. The reaction medium is stirred at −78° C. for 30 minutes. 100 μl of acetic acid are added and the mixture is brought back to ambient temperature. After purification by chromatography (90 CH2Cl2/10 EtOH) and recrystallization from EtOH/Et2O, 324 mg of the title product are obtained: MS (SI) m/z 342 (M++1).

4-Amino-8-(2′-deoxy-β-D-ribofuranosyl)pyrazolo[1,5-a]-1,3,5-triazine (Ib29). By replacing, in example Ib4, the 8-(2-chlorobenzoyl)-4-(N-methyl-N-phenylamino)-2-n-propylpyrazolo[1,5-a]-1,3,5-triazine (Ib3) with 8-(2′-deoxy-β-D-ribofuranosyl)-4-(N-methyl-N-phenylamino)pyrazolo[1,5-a]-1,3,5-triazine (Ib27) and the methylamine with an ammonia-saturated ethanol solution, the title product is obtained, in the same way, in the form of a white powder (yield: 63%).

4-Amino-8-(2′-deoxy-β-D-xylofuranosyl)pyrazolo[1,5-a]-1,3,5-triazine (Ib30). By replacing, in example Ib29, the 8-(2′-deoxy-β-D-ribofuranosyl)-4-(N-methyl-N-phenylamino)pyrazolo[1,5-a]-1,3,5-triazine (Ib27) with 8-(2′-deoxy-β-D-xylofuranosyl)-4-(N-methyl-N-phenylamino)pyrazolo[1,5-a]-1,3,5-triazine (Ib28), the title product is obtained, in the same way, in the form of a white powder (yield: 56%).

### EXAMPLE 3

### Synthesis of the Compounds of Formula Ia

8-Benzyl-2-methylpyrazolo[1,5-a]-1,3,5-triazin-4-one (Ia1). A solution of 300 mg of 8-benzyl-2-methyl-4-(N-methyl-N-phenylamino)pyrazolo[1,5-a]-1,3,5-triazine and 100 mg of NaOH in 10 ml of an H2O/EtOH (2:8) mixture is stirred at ambient temperature for 12 hours. It is evaporated to dryness. 3 ml of H2O are added, and the reaction medium is neutralized with 1N HCl (pH=6-7). The reaction medium is filtered and washed with a minimum of H2O. The product is obtained in the form of colorless crystals (yield: 68%). Mp: 225° C. 1H-NMR (200 MHz, DMSO-d6): 2.35 (s, 3H, CH3), 3.90 (s, 2H, CH2), 7.14-7.34 (m, 5H, Ph), 7.91 (s, H7 pyrazole), 12.39 (broad s, 1 exchangeable H, NH).

3-(4-Oxopyrazolo[1,5-a]-1,3,5-triazin-8-yl)propionic acid (Ia2). A solution of 700 mg of ethyl 3-[4-(N-methyl-N-phenylamino)pyrazolo[1,5-a]-1,3,5-triazin-8-yl]propionate (Ib6), and 300 mg of sodium hydroxide in a mixture of 700 μl of H2O and of 6 ml of ethanol is refluxed for 15 minutes. The reaction medium is cooled to 0° C. The crystals obtained are filtered off. They are dissolved in 7 ml of H2O and acidified to pH 2 with concentrated hydrochloric acid. The solution is stirred at 0° C. for 5 minutes. The crystals formed are filtered off. They are washed twice with 1 ml of H2O, once with 1 ml of EtOH and twice with 10 ml of Et2O. The product is recrystallized from EtOH/Et2O. 480 mg of the title product are obtained in the form of colorless crystals. Mp=277° C. 1H-NMR (300 MHz, DMSO-d6): 2.6 (t, J=7.5 Hz, 2H, CH2), 2.80 (t, J=7.5 Hz, 2H, CH2), 7.97 (s, 2H, 2 CH), 12.1 (broad s, 1H, OH), 12.4 (broad s, 1H, OH).

Ethyl 3-[4-oxopyrazolo[1,5-a]-1,3,5-triazin-8-yl]-acrylate (Ia3). A solution of ethyl 3-[4-(N-methyl-N-phenylamino)pyrazolo[1,5-a]-1,3,5-triazin-8-yl]acrylate (Ib5, 200 mg) and of 60 mg of NaOH in a 1:9 H2O/EtOH mixture is heated at 50° C. for 10 minutes. The reaction medium is evaporated to dryness. 15 ml of H2O are added and the pH is brought to 7-8 with a 0.1N HCl solution. Extraction is carried out 3 times with 30 ml of EtOAc. Purification is carried out by chromatography on silica (4 EtOAc, 4 CH2Cl2, 1 EtOH). The product is recrystallized from EtOH/Et2O. The title product is obtained in the form of colorless crystals (yield: 27%). Mp: 253° C. 1H-NMR (300 MHz, DMSO-d6): 1.23 (t, J=7.1, 3H, CH3), 4.15 (q, J=7.1, 2H, CH2), 6.65 (d, J=16.1, 1H, CH), 7.60 (d, J=16.1, 1H, CH), 8.17 (s, 1H, CH), 8.49 (s, 1H, CH).

Sodium 4-[(hydroxy)[4-oxopyrazolo[1,5-a]-1,3,5-triazin-8-yl]methyl]benzoate (Ia4). By replacing, in example Ia2, the ethyl 3-[4-(N-methyl-N-phenylamino)pyrazolo[1,5-a]-1,3,5-triazin-8-yl]propionate (Ib6) with methyl 4-[(hydroxy)[4-(N-methyl-N-phenylamino)]pyrazolo[1,5-a]-1,3,5-triazin-8-yl]methyl]benzoate (Ib1), the title product (yield: 82%) is obtained, after salification of the carboxylic acid function with sodium hydroxide. Mp>300° C. 1H-NMR (300 MHz, DMSO-d6): 5.42 (broad s, 1H, NH), 5.84 (s, 1H, CH), 7.27-7.47 (m, 3H, 3 ArH), 7.71-7.79 (m, 3H, 3 CH).

Sodium 4-[[1-(oxo)-3-(4-oxopyrazolo[1,5-a]-1,3,5-triazin-8-yl)propyl]amino]benzoate (Ia5). By replacing, in example Ia2, the ethyl 3-[4-(N-methyl-N-phenylamino)pyrazolo[1,5-a]-1,3,5-triazin-8-yl]propionate (Ib6) with methyl 4-[[1-oxo-3-[4-(N-methyl-N-phenylamino)pyrazolo[1,5-a]-1,3,5-triazin-8-yl]-propyl]amino]benzoate (Ib8), the title product (yield=82%) is obtained in the same way. 1H-NMR (300 MHz, D2O): 2.98 (t, J=7.2, 2H, CH2), 3.30 (t, J=7.2, 2H, CH2), 7.60 (d, J=8.50, 2H, 2 ArH), 8.08 (d, J=8.50, 2H, 2 ArH), 8.13 (s, 1H, 1 ArH), 8.17 (s, 1H, 1 ArH). MS: 328 (M+H)+.

8-Benzoyl-2-methylpyrazolo[1,5-a]-1,3,5-triazin-4-one (Ia6). By replacing, in example Ia2, the 8-benzoyl-2-methyl-4-(N-methyl-N-phenylamino)pyrazolo[1,5-a]-1,3,5-triazine acid (Ib9) with ethyl 3-[4-(N-methyl-N-phenylamino)pyrazolo[1,5-a]-1,3,5-triazin-8-yl]propionate (Ib6), the title product (yield=92%) is obtained in the same way.

N-[2-(3,4-Dihydroxyphenyl)ethyl]-3-(4-oxopyrazolo-[1,5-a]-1,3,5-triazin-8-yl)propionamide (Ia7). By replacing, in example Ia3, the ethyl 3-[4-(N-methyl-N-phenylamino)pyrazolo[1,5-a]-1,3,5-triazin-8-yl]acrylate (Ib5) with N-[2-(3,4-dihydroxyphenyl)ethyl]-3-[4-(N-methyl-N-phenylamino)pyrazolo[1,5-a]-1,3,5-triazin-8-yl]propionamide (Ib22), the title product (yield=91%) is obtained in the same way: MS (SI) m/z 344 (M++1).

3-[4-Oxopyrazolo[1,5-a]-1,3,5-triazin-8-yl)-N-[3-(2-oxopyrrolidin-1-yl)propyl]propionamide (Ia8). A solution of 5N NaOH (0.28 ml, 1.42 mmol) is added to a solution of 3-[4-(N-methyl-N-phenylamino)pyrazolo[1,5-a]-1,3,5-triazin-8-yl)-N-[3-(2-oxopyrrolidin-1-yl)propyl]propionamide (Ib23) (120 mg, 0.28 mmol) in ethanol (10 ml). The solution is stirred for 5 h at ambient temperature. The solvents are evaporated off. The residue obtained is purified by column chromatography on silica gel (eluent: CH2Cl2/MeOH, 85:15) so as to give the title compound (62 mg, 66%, solid); Mp=180-181° C. (methanol). 1H-NMR (300 MHz, DMSO-d6) δ 1.50-1.57 (m, 2H, CH2), 1.84-1.94 (m, 2H, CH2), 2.19 (t, 2H, J=7.9 Hz, CH2), 2.38 (t, 2H, J=7.6 Hz, CH2), 2.78 (t, 2H, J=7.6 Hz, CH2), 2.99 (broad q, 2H, J=6.4 Hz, CH2), 3.10 (t, 2H, J=7.1 Hz, CH2), 3.28 (t, 2H, J=7.1 Hz, CH2), 7.80 6.73 (broad t, 1H, J=5.9 Hz, NH), 7.88 (s, 1H, HAr), 7.93 (s, 1H, HAr); 13C-NMR (75 MHz, DMSO-d6) d 17.5 (CH2), 18.5 (CH2), 26.9 (CH2), 30.4 (CH2), 35.7 (CH2), 36.2 (CH2), 39.5 (CH2), 46.3 (CH2), 111.3 (C), 145.1 (CH), 145.8 (C), 171.1 (2 CO), 173.8 (CO); MS (SI) m/z 333 (M++1).

N-[2-Hydroxy-2-(3,4-dihydroxyphenyl)ethyl]-3-[4-oxo-pyrazolo[1,5-a]-1,3,5-triazin-8-yl]propionamide (Ia9). By replacing, in example Ia3, the ethyl 3-[4-(N-methyl-N-phenylamino)pyrazolo[1,5-a]-1,3,5-triazin-8-yl]acrylate (Ib5) with N-[2-hydroxy-2-(3,4-dihydroxyphenyl)ethyl]-3-[4-(N-methyl-N-phenylamino)pyrazolo[1,5-a]-1,3,5-triazin-8-yl]propionamide (Ib24), the title product (yield=83%) is obtained in the same way: MS (SI) m/z 360 (M++1).

### EXAMPLE 4

### Pharmacological Activity: Stimulation of the Synthesis of Neurotrophic Factors

Compounds according to the invention were evaluated for their neurotrophic properties. The idea is therefore to observe the behavior of a neuron cell culture in the absence and presence of such molecules. The molecule called Ia5 used during this example is a molecule having the general structure Ic1, where n=2 and m=0, in the form of a sodium salt.

Preparation of Neurons

Rats of the Sprague Dawley strain are raised in the laboratory up to adult age, i.e. three months after their birth. They are fed ad libitum in rooms at a temperature of 22±2° C., where the light cycle is 12 hours of light (day) and 12 hours of dark.

The adult animals are mated and the female rats are separated the following day. After 16 days, the gestating rats undergo a cesarean and the fetuses are placed in a Petri dish 100 mm in diameter. They are transferred, under a laminar flow hood, into sterile medium. The fetuses are isolated by units and are dissected under a binocular magnifying lens in sterile medium. The cerebral cortex is isolated and placed in a tube containing Neurobasal medium without antibiotic. The tissue is dissected by suctioning back and forward into single cells in a volume of 2 ml. The cell suspension is then carefully deposited onto 2 ml of inactivated fetal calf serum. The tube is centrifuged at low gravity (800 g) for 5 min at ambient temperature. The cell pellet is recovered and the cells are resuspended in complete Neurobasal medium. The cells are counted using a Mallassez hematometer in the presence of trypan blue in order to determine the cell viability. The culturing takes place by adding 800 000 cells to Petri dishes 60 mm in diameter containing the complete Neurobasal medium preheated and equilibrated beforehand in a CO2 incubator. These dishes were precoated with a layer of polylysine the day before the manipulation. The temperature of the incubator is regulated at 37° C., the CO2 level at 5% and the humidity is saturating. The Petri dishes containing the cells are then placed in the incubator.

Approximately two hours after being placed in culture, the cells which were refringent straight after seeding become black, which is a sign of adhesion to the bottom of the Petri dish. Twenty-four hours after the placing in culture, the neurites begin to grow. Growth continues for about ten days, and then signs of senescence begin to appear. These cultures constitute primary neuron cultures.

Neuron Treatments

The neuron cultures as prepared above are used as controls. 5 dishes will be used in order to have a statistical approach.

In the other dishes, the test molecule is added at various concentrations: 0.1 μmol/l, 1 μmol/l and 10 μmol/l. In each case, the manipulation is repeated 5 times.

The neurons are examined under a phase-contrast inverted microscope (Zeiss Axiovert 135) every day after seeding.

The neurons are photographed at various magnifications using a photographic device, and compared between series.

Results

The presence of the molecule Ia5 on the neurons results in greater neurite development than in the cells acting as control. A thickening and an elongation of the neurites is observed in B compared with the control A (FIG. 1).

It is also noted that the addition of astrocyte culture supernatant contributes to increasing the density of neurites in the presence of the molecule, compared with the control.

### EXAMPLE 5

### Cyclic Nucleotide Phosphodiesterase Inhibition

Determination of PDE4 Inhibition

This new family of compounds was tested as an inhibitor of human phosphodiesterase type 4 (source: U-937) by following the method described by Torphy, T. J., Zhou, H. L. and Cieslinski, L. B. (J. Pharmacol. Exp. Ther., 1992, 263, 1195-1205). The concentration of substance which inhibits the enzymatic activity by 50% (IC50) was determined at a substrate ([3H]cAMP+cAMP) concentration equal to 1 μM, the incubation time being 30 minutes at 30° C. A quantitative measurement of the hydrolysis product [3H]-5′-AMP was determined by scintillation. The compounds are compared to the control rolipram, which, in this test, has an IC50 of 0.39 μM. The most powerful compounds according to the invention have an IC50 of between 20 nM and 0.01 nM.

Determination of PDE2 Inhibition

This novel family of compounds was tested as an inhibitor of human phosphodiesterase type 2 (source: U-937 cells) by following the method described by Torphy, T. J., Zhou, H. L. and Cieslinski, L. B. (J. Pharmacol. Exp. Ther., 1992, 263, 1195-1205). The concentration of substance which inhibits the enzymatic activity by 50% (IC50) was determined at a substrate ([3H]cAMP+cAMP) concentration equal to 1 μM, the incubation time being 30 minutes at 30° C. A quantitative measurement of the hydrolysis product [3H]-5′-AMP was determined by scintillation. The compounds are compared to the control EHNA, which, in this test, has an IC50 of 2.1 μM. The most powerful compounds according to the invention have an IC50 of between 5 μM and 1 nM.

Determination of the selectivity with respect to PDE1, 3, 5 and 6

The compounds most active on PDE2 and/or PDE4 were tested for their selectivity with respect to the following

cyclic nucleotide phosphodiesterases: PDE1 (bovine), PDE3 (human), PDE5 (human) and PDE6 (bovine), by following the methods described, respectively, by: (i, PDE1) Nicholson C. D., JACKMAN S. A. and WILKE R. (Brit. J. Pharmacol. 1989, 97, 889-897); (ii, PDE3 and PDE5) Weishaar, R. E., Burrows, S. D., Kobylarz, D. C., Quade, M. M. and Evans, D. B. (Biochem. Pharmacol., 1986, 35, 787-800); (iii PDE6) Ballard, A. S., Gingell, C. J., Tang, K., Turner, L. A., PRICE, M. E. (J. Urol, 1998, 159, 2164-2171). The concentration of substance which inhibits the enzymatic activity by 50% (IC50) was determined for PDE1 and PDE3 at a substrate ([3H]cAMP+cAMP) concentration equal to 1 μM, the incubation time being 30 minutes at 30° C. In the case of PDE5 and PDE 6, the substrate used is ([3H]cGMP+cGMP) at a concentration of 1 μM for PDE5 and 2 μM for PDE6. A quantitative measurement of the hydrolysis products [3H]-5′-AMP and [3H]-5′-GMP was determined by scintillation. The compounds are compared to the following controls: 8-methoxy-IBMX (IC50=2.9 μM) for PDE1, milrinone (IC50=0.25 μM) for PDE3, dipyridamole (IC50=0.5 μM) for PDE5, and zaprinast (IC50=0.38 μM) for PDE6.

The preferred molecules according to the invention exhibit an excellent potency and selectivity profile with respect to phosphodiesterase type 4 or to phosphodiesterase type 2, insofar as these compounds inhibit the other PDEs, in particular PDE3, more weakly. The selectivity coefficient is, for the most potent compounds, greater than 100. Ideally, this coefficient is greater than 1000 or 10 000 for the most potent compounds of the invention. In certain cases, molecules having similar activities for PDE2 and PDE4 were obtained. These compounds are, on the other hand, selective with respect to the other types of PDE (PDE1, 3, 5 and 6).

### EXAMPLE 7

### Anti-Inflammatory Properties of the Compounds of the Invention

The compounds according to the invention were evaluated for their anti-inflammatory properties on venous blood mononuclear cells (PBMCs). More particularly, the cells were incubated for 24 hours in the presence of the molecule tested, after activation with lipopolysaccharide (LPS) (1 μg/ml) according to the protocol described by Schindler, R., Mancilla, J., Endres, S., Ghorbani, R., Clark, S. C. and Dinarello, C. A. (Blood, 1990, 75, 40-47). After incubation, the TNFα concentrations were measured in the culture supernatants by the EIA method. The compounds were compared with the control dexamethasone, which, in this test, has an IC50 of 4.6 μM. The most potent compounds according to the invention have an IC50 of less than 1 μM, i.e. they are notably more active than dexamethasone. Some compounds of the invention have an IC50 of between 100 nM and 1 nM on this test.

### EXAMPLE 8

### Neuroprotective Effect on Models of Induced Apoptosis

Neuroprotective effect on a model of apoptosis induced by elimination of BDNF

This test was carried out according to the protocol described by Estevez A. G. et al. (J. Neurosci. 1998, 18(3), 923-931). Briefly, when primary cultures of rat embryonic motoneuron cells are deprived of brain-derived neurotrophic factor (BDNF), an induction of neuronal nitric oxide synthase (NOS) was observed, resulting in the gradual death of the neurons by apoptosis: between 18 and 24 hours after having realized the biological preparation, more than 60% of the neurons die. In this model of induced apoptosis, the compound sodium 4-[[1-(oxo)-3-(4-oxopyrazolo[1,5-a]-1,3,5-triazin-8-yl)propyl]amino]benzoate (Ia5) protects more than 70% of the neurons at a concentration of 1 mM.

Neuroprotective effect on a model of motoneuron apoptosis induced by peroxynitrite

This test was carried out according to the protocol described by Cassina P. et al. (J. Neurosci. Res. 2002 67(1): 21-9). Briefly, the oxidative stress mediated by nitric oxide and its toxic metabolite, peroxynitrite, has been associated with motoneuron degeneration, in particular in amyotrophic lateral sclerosis. The astrocytes of the spinal cord respond to extracellular concentrations of peroxynitrite by adopting a phenotype which is cytotoxic for the motoneurons. In this model of apoptosis induced by peroxynitrite, the compound sodium 4-[[1-(oxo)-3-(4-oxopyrazolo[1,5-a]-1,3,5-triazin-8-yl)propyl]amino]benzoate (Ia5) protects more than 60% of the neurons at a concentration of 1 mM.

1. A compound wherein said compound having a structure represented by formula (Ia), 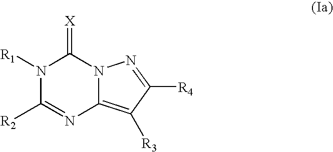
or formula (Ib), 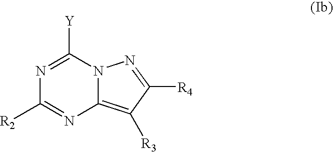
wherein R1 represents:
a hydrogen atom,
or a (C1-C12)alkyl, (C3-C6)cycloalkyl, (C6-C18)aryl, (C6-C18)aryl(C1-C4)alkyl, (C1-C12)alkyl(C6-C18)aryl, (C2-C8)alkenyl, (C2-C8)alkynyl, (C1-C8)alkoxy or hydroxyl group,
or an aromatic or nonaromatic (C5-C18)heterocycle containing from 1 to 3 hetero atoms and being attached directly to the nitrogen atom in the 1-position by means of a single bond or by means of a (C1-C6)alkyl, (C2-C6)alkenyl or (C2-C6)alkynyl group,
or a group NR′R″ or NHCOR′R″, R′ and R″, independently of one another, selected from the group consisting of a hydrogen atom, (C1-C6)alkyl, (C3-C6)cycloalkyl and (C6-C12)aryl groups, and aromatic or nonaromatic (C5-C12)heterocycles containing from 1 to 3 hetero atoms;
R2 represents:
a hydrogen atom,
a halogen atom,
a group: (C1-C6)alkoxy, (C1-C10)alkyl, (C1-C6)-alkylCOOH, (C1-C6)alkylCOONa, perfluoro(C1-C6)alkyl, (C3-C6)cycloalkyl, acyl, (C2-C6)alkenyl, (C2-C6)alkynyl, (C6-C18)aryl, (C6-C18)arylCOOH, (C6-C18)arylCOONa, (C6-C18)aryl(C1-C4)alkyl, (C1-C6)alkyl(C6-C18)aryl, (C5-C18)heteroaryl, (C1-C6)alkyl(C5-C18)heteroaryl, (C2-C6)alkenyl(C5-C18)heteroaryl, (C2-C6)alkynyl(C5-C18)heteroaryl, CH(OH)(C6-C18)aryl, CO(C6-C18)aryl, (CH2)nCONH—(CH2)m-(C6-C18)aryl, (CH2)nSO2NH—(CH2)n-(C6-C18)aryl or (CH2)nCONH—CH(COOH)—(CH2)p-(C6-C18)aryl with n=1 to 4, m=0 to 3 and p=0 to 2, in which one or more groups —CH2- can be optionally replaced with —O—, —S—, —S(O)—, —S(O)2- or —NH—, and can be optionally substituted with one or more radicals chosen from the following radicals: (C1-C6)alkyl, hydroxyl, oxo, (C6-C18)aryl(C1-C8)alkyl, (C6-C18)aryl, halogen, cyano, phosphate, alkylphosphate, nitro, alkoxy, (C5-C18)heteroaryl, (C5-C18)heteroaryl(C1-C6)alkyl, COOH, CONRxRy, NRxCONHRy, ORx, SRx, SORx, SO2Rx, CORx, COORx, NRxSO2Ry or NRxRy in which
(i) Rx and Ry, independently of one another, are chosen from a hydrogen atom and the following groups: (C1-C6)alkyl, (C3-C6)cycloalkyl, (C6-C18)aryl, (C6-C18)aryl(C1-C4)alkyl, (C1-C12)alkyl(C6-C18)aryl, (C3-C6)cycloalkyl(C6-C12)aryl, (C1-C6)alkoxy(C1-C6)alkyl, (C5-C12)heteroaryl containing 1 to 3 hetero atoms, OR′, NR′R″ and NHCOR′R″, R′ and R″, independently of one another, being chosen from a hydrogen atom, (C1-C6)alkyl, (C3-C6)cycloalkyl and (C6-C12)aryl groups, and aromatic or nonaromatic (C5-C12)heterocycles containing 1 to 3 hetero atoms, or
(ii) Rx and Ry together form a linear or branched hydro-carbon-based chain having from 2 to 6 carbon atoms, optionally containing one or more double bonds and/or optionally interrupted with an oxygen, sulfur or nitrogen atom,
or a nitro, cyano, ORx, SRx, SORx, SO2Rx, CORX, CONRXRy, COORx, NRxCORy, NRxSO2Ry or NRxRy group in which Rx and Ry are as defined above; and
R₃ represents:
a hydrogen atom,
a halogen atom,
a group: (C₁-C₆)alkoxy, (C₁-C₁₀)alkyl, (C₁-C₆)alkylCOOH, (C₁-C₆)alkylCOONa, perfluoro (C₁-C₆)alkyl, (C₃-C₆) cycloalkyl, acyl, (C₂-C₆)alkenyl, (C₂-C₆)alkynyl, (C₁-C₆)alkyl(C₆-C₁₈)aryl, (C₁-C₆)alkyl(C₅-C₁₈) heteroaryl, (C₂-C₆)alkenyl(C₅-C₁₈) heteroaryl, (C₂-C₆)alkynyl(C₅-C₁₈) heteroaryl, CH(OH)(C₆-C₁₈)aryl, CO(C₆-C₁₈)aryl, (CH₂)ₙCONH— (CH₂)ₘ—(C₆-C₁₈)aryl, (CH₂)ₙSO₂NH—(CH₂)ₘ—(C₆-C₁₈)aryl or (CH₂)ₙCONH—CH(COOH)— (CH₂)ₚ—(C₆-C₁₈)aryl with n=1 to 4, m=0 to 3 and p=0 to 2, in which one or more groups —CH₂— can be optionally replaced with —O—, —S—, —S(O)—, —S(°)₂— or —NH—, and can be optionally substituted with one or more radicals chosen from the following radicals: (C₁-C₆)alkyl, hydroxyl, oxo, (C₆-C₁₈)aryl(C₁-C₈)alkyl, (C₆-C₁₈)aryl, halogen, cyano, phosphate, alkylphosphate, nitro, alkoxy, (C₅-C₁₈)heteroaryl, (C₅-C₁₈)heteroaryl(C₁-C₆)alkyl, COOH, CONRxRy, in which
(i) Rₓ and R_{y}, independently of one another, are chosen from a hydrogen atom and the following groups: (C₁-C₆)alkyl, (C₃-C₆) cycloalkyl, (C₆-C₁₈)aryl, (C₆-C₁₈)aryl(C₁-C₄)alkyl, (C₁-C₁₂)alkyl(C₆-C₁₈)aryl, (C₃-C₆) cycloalkyl(C₆-C₁₂)aryl, (C₁-C₆)alkoxy(C₁-C₆)alkyl, (C₅-C₁₂)heteroaryl containing 1 to 3 hetero atoms, OR′, NR′R″ and NHCOR′R″, R′ and R″, independently of one another, being chosen from a hydrogen atom, (C₁-C₆)alkyl, (C₃-C₆)cycloalkyl and (C₆-C₁₂)aryl groups, and aromatic or nonaromatic (C₅-C₁₂)heterocycles containing 1 to 3 hetero atoms, or
(ii) Rₓ and R_{y} together form a linear or branched hydrocarbon-based chain having from 2 to 6 carbon atoms, optionally containing one or more double bonds and/or optionally interrupted with an oxygen, sulfur or nitrogen atom, or a nitro, cyano, ORₓ, SRₓ, SORₓ, SO₂Rₓ, CORₓ, CONRₓR_{y}, COORₓ, NRₓCOR_{y}, NRₓSO₂R_{y} or NRₓR_{y} group in which Rₓ and R_{y} are as defined above; and
wherein the “aryl” groups of groups R2 and R3 can be replaced with aromatic or nonaromatic C4-C10 “heterocycles” containing from 1 to 3 hetero atoms;
X represents either an oxygen or sulfur atom, or a group NRₓ in which Rₓ is as defined above; and
Y represents either a halogen atom, or a (C₁-C₆)alkyl, (C₂-C₆)alkenyl, (C₂-C₆)alkynyl, (C₁-C₆)alkoxy, (C₃-C₆) cycloalkyl, ORₓ, SRₓ, SORₓ, SO₂Rₓ, NRₓCOR_{y}, NRₓSO₂R_{y} or NRₓR_{y} group in which Rₓ and R_{y} are as defined above and R₁ is not present;
R4 represents:
a hydrogen atom, it being possible for said formulae (Ia) and (Ib) to be, with respect to one another, tautomeric forms according to the definition of R1, of X and of Y, its tautomeric forms, its isomers, diastereoisomers and enantiomers, and its pharmaceutically acceptable base or acid addition salts, with the proviso that:
when Y, in formula (Ib), represents ORx, then Rx is necessarily different from aryl and aralkyl;
when simultaneously, in formula (Ib), Y represents NRxRy and Rx represents H, then Ry is necessarily different from aryl and aralkyl; except when R₃ is (C₁-C₁₀)alkyl, perfluoro(C₁-C₆)alkyl, or (C₃-C₆)cycloalkyl;
when Y, in formula (Ib), represents a group NRxRy in which at least one of the groups Rx or Ry is chosen from optionally substituted phenyl or pyridyl groups, then R3 is different from a (C1-C10)alkyl, (C2-C10)alkenyl, (C2-C10)alkynyl, (C3-C8)cycloalkyl and (C3-C6)cycloalkyl(C1-C4)alkyl group, it being possible for the latter to be optionally substituted;
when simultaneously, in formula (Ib), Y represents a methylamino, benzylamino, pyrrolidinyl, dimethylamino or 1-piperazinyl group and R2 represents methyl or n-propyl, then R3 is different from iodo and benzoyl;
when simultaneously, in formula (Ib), Y represents NHCH₃, R2 represents CH₃ and R4 represents a hydrogen atom, then R3 is different from benzyl, (2-naphthyl)methyl, pentyl, benzoyl, propyne, penten-1-yl, 2-chlorobenzoyl, 2-methylaminobenzoyl, 4-methoxybenzoyl, 3-trifluoromethylbenzoyl, furfuryl, (3-furyl)methyl, (2-thienyl)methyl, 2-hydroxypropyl, iodo, nitro, acetylamino, benzoylamino and diethylaminocarbonyl;
when simultaneously, in formula (Ib), Y represents NHCH₃, R4 represents H and R3 represents benzoyl or iodo, then R2 is different from methyl, ethyl, n-propyl, n-butyl, thiomethyl, methoxymethyl, phenyl and 2-furyl;
when simultaneously, in formula (Ib), Y represents NHCH₃, R4 represents H and R3 represents benzyl or 2-methoxybenzyl, then R2 is different from methyl, n-propyl and trifluoromethyl;
when simultaneously, in formula (Ib), Y represents a methylamino, benzylamino, pyrrolidinyl, dimethylamino or 1-piperazinyl group and R2 represents methyl or n-propyl, then R3 is different from iodo and benzoyl;
when simultaneously, in formulae (Ia) and (Ib), R1 is a hydrogen atom with R2 chosen from CH3, C2H5 or C6H5, R3 is chosen from H, COOEt, Cl, I or Br, and R4 represents H, then Y is different from H, OH, CH3, C2H5, C6H5, n-C3H7, iso-C3H7, SH, SCH3, NH(n-C4H9) or N(C2H5)2 and X is different from O;
when simultaneously, in formula (Ib), R1 represents H, R3 represents Br or H, and R2 is chosen from H, CH3 or SCH3, and R4 is H, then Y is different from SCH3, NH(n-Pr), NH(n-Bu), N(Et)2, piperidyl, OH, SH, O(i-Pr), CH3, SEt, OCH3 and O(n-Pr);
when simultaneously, in formula (Ib), R2 represents CF3, CH3OCH2-, Ph, Et, n-Pr or CH3, and Y represents NHCH3, N(CH3)2 or N(CH3)Ph, and R4=H then R3 is different from β-D-glycero-pentofuran-3′-ulos-1′-yl, 2′-deoxy-β-D-ribofuranosyl, 2′-deoxy-β-D-xylofuranosyl, 2′-deoxy-β-D-ribofuranosyl-3′,5′-bis(dibenzyl phosphate), cyclic benzyl 2′-deoxy-β-D-xylofuranosyl-3′,5′-phosphate, 2′-deoxy-β-D-ribofuranosyl-3′,5′-bisphosphate and cyclic 2′-deoxy-β-D-xylofuranosyl-3′,5′-phosphate;
and said compound does not correspond to the following formulae:

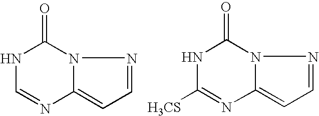

2. The compound of claim 1 wherein:
R1 represents a hydrogen atom or a (C1-C12)alkyl group;
R2 represents a hydrogen or sulfur atom, or a (C1-C6)alkyl group, or a trifluoro(C1-C6)alkyl group, or an amino group, or a group SRx where Rx is as defined above;
R3 represents a hydrogen atom, or a halogen atom, or a nitro, (C1-C6)alkyl, trifluoro(C1-C6)alkyl, acyl, (C2-C6)alkenyl, (C2-C6)alkynyl, (CH2)nCONH—(CH2)maryl, (CH2)nSO2NH—(CH2)maryl or (CH2)nCONH—CH(COOH)—(CH2)paryl group with n=1 to 4, m=0 to 3 and p=0 to 2, or a group NR′R″ or NHCOR′R″, R1 and R″, independently of one another, being chosen from a hydrogen atom, (C1-C6)alkyl, (C3-C6)cycloalkyl and (C6-C12)aryl groups, and aromatic or nonaromatic (C5-C12)heterocycles containing 1 to 3 hetero atoms;
R4 represents a hydrogen atom;
X represents an oxygen or sulfur atom; and
Y represents either a halogen atom, or a (C1-C6)alkyl, (C2-C6)alkynyl, phenyl, ORx, SRx or NRxRy group.

3. The compound of claim 1 wherein:
R1 represents a hydrogen atom or a methyl group;
R2 represents a hydrogen or sulfur atom, or a methyl, propyl, trifluoromethyl, amino or thiomethyl group;
R3 represents an iodine atom, or an amino, nitro, acyl-amino, benzyl, 2-methoxybenzyl, furfuryl, 3-furylmethyl, 2-thienylmethyl, 3-thienylmethyl, 2-pyridylmethyl, 2-chlorobenzoyl —CH2CH2COOH, CH2CH2COONa, C6H4COOH, C6H4COONa, CH2=CHCOOC2H5, propyn-1-yl, (CH2)2CONH—C6H4COONa, (CH2)CONH—(CH2)2-indole, (CH2)2CONH—CH(COOH)(CH2)indole, (CH2)CONH—(CH2)2C6H40H or (CH2)2CONH—CH2C6H40H group;
X represents an oxygen atom; and
Y represents an OH, SH, N-methyl-N-phenylamino (NPhCH3), N-methyl-N-(4-acylaminophenyl)amino or triazole group.

4. The compound of claim 1 wherein said compound has a structure represented formulae (Ic1) and (Ic2) 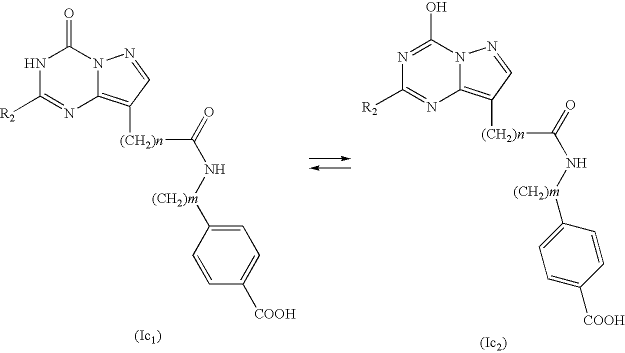
or its pharmaceutically acceptable base or acid addition salts, wherein n=1 to 4, and m=0 to 2.

5. The compound of claim 4 wherein R2 represents a hydrogen atom, n=1 and m=0.

6. Sodium 4-[[1-(oxo)-3-(4-oxopyrazolo[1,5-a]-1,3,5-triazin-8-yl)propyl]amino]benzoate.

7. The compound of claim 1 wherein Y represents a methylamino or cyclopropylamino group;
R2 represents an iodine or sulfur atom, or a methyl, propyl, cyclopropyl, perfluoroethyl, perfluoropropyl, trifluoromethyl, allyl, trifluoromethylvinyl, vinyl, 1-propynyl or ethynyl group;
R3 is selected from the group consisting of an iodine atom, and a benzyl, 2-methoxybenzyl, 2-fluorobenzyl, 2-bromobenzoyl, furfuryl, 2-furylcarbonyl, 3-furylmethyl, 2-thienylmethyl, 3-thienylmethyl, 2-pyridylmethyl, 2-chlorobenzoyl, cyclopentyl or cyclohexyl group; and
R4 represents a hydrogen atom.

8. The compound of claim 1 wherein X represents an oxygen atom;
Y represents an OH or NH2 group;
R1 represents a hydrogen atom or optionally an alkyl group having from 1 to 3 carbons;
R3 represents a hydrogen atom or a substituted benzyl group
R4 represents a hydrogen atom.

9. The compound of claim 1 wherein said compound is selected from the group consisting of:
8-Iodo-4-(N-methyl-N-phenylamino)pyrazolo[1,5-a]-1,3,5-triazine;
8-Iodo-4-[N-methyl-N-(4-nitrophenyl)amino]pyrazolo[1,5-a]-1,3,5-triazine;
8-Iodo-4-(triazol-4-yl)pyrazolo[1,5-a]-1,3,5-triazine;
8-Acetamido-2-methylpyrazolo[1,5-a]-1,3,5-triazin-4-one;
Methyl 4-[(hydroxy)[4-(N-methyl-N-phenylamino)pyrazolo[1,5-a]-1,3,5-triazin-8-yl]methyl]benzoate;
8-[(2-Chlorophenyl)(hydroxy)methyl]-4-(N-methyl-N-phenylamino)-2-n-propylpyrazolo[1,5-a]-1,3,5-triazine;
8-(2-Chlorophenyl)-4-(N-methyl-N-phenylamino)-2-n-propylpyrazolo[1,5-a]-1,3,5-triazine;
8-(2-Chlorophenyl)-4-(N-methylamino)-2-n-propylpyrazolo[1,5-a]-1,3,5-triazine;
Ethyl 3-[4-(N-methyl-N-phenylamino)pyrazolo[1,5-a]-1,3,5-triazin-8-yl]acrylate;
Ethyl 3-[4-(N-methyl-N-phenylamino)pyrazolo[1,5-a]-1,3,5-triazin-8-yl]propionate;
3-[4-(N-Methyl-N-phenylamino)pyrazolo[1,5-a]-1,3,5-triazin-8-yl]propionic acid;
Methyl 4-[[1-oxo-3-[4-(N-methyl-N-phenylamino)pyrazolo[1,5-a]-1,3,5-triazin-8-yl]propyl]amino]benzoate;
4-(Cyclopropylamino)-8-(2-fluorobenzoyl)-2-methylpyrazolo[1,5-a]-1,3,5-triazine;
Ethyl 4-(N-methyl-N-phenylamino)pyrazolo[1,5-a]-1,3,5-triazine-8-carboxylate;
tert-Butyl 3-[4-(N-methyl-N-phenylamino)pyrazolo[1,5-a]-1,3,5-triazin-8-yl]acrylate;
tert-Butyl 3-[4-(N-methyl-N-phenylamino)pyrazolo[1,5-a]-1,3,5-triazin-8-yl]propionate;
4-(N-Methyl-N-phenylamino)-8-(β-D-glycero-pentofuran-3′-ulos-1′-yl)pyrazolo[1,5-a]-1,3,5-triazine;
8-[(3-Furyl)(hydroxy)methyl]-4-(N-methyl-N-phenylamino)-2-n-propylpyrazolo[1,5-a]-1,3,5-triazine;
8-(3-Furylmethyl)-2-n-propyl-4-(N-methyl-N-phenylamino)pyrazolo[1,5-a]-1,3,5-triazine;
2-Trifluoromethyl-8-(3-furylmethyl)-4-(cyclopropylamino)pyrazolo[1,5-a]-1,3,5-triazine;
2-Thiomethyl-8-(3-furylmethyl)-4-(N-methylamino)-pyrazolo[1,5-a]-1,3,5-triazine;
8-(3-Furylmethyl)-4-(N-methylamino)-2-n-propylpyrazolo[1,5-a]-1,3,5-triazine;
2-Trifluoromethyl-8-cyclopentyl-4-(N-methylamino)-pyrazolo[1,5-a]-1,3,5-triazine;
2-Pentafluoroethyl-8-(2-methoxybenzyl)-4-(N-methylamino)pyrazolo[1,5-a]-1,3,5-triazine;
4-(N-Cyclopropylamino)-2-trifluoromethyl-8-(2-methoxybenzyl)pyrazolo[1,5-a]-1,3,5-triazine;
4-(N-Cyclopropylamino)-8-(2-methoxybenzyl)-2-n-propylpyrazolo[1,5-a]-1,3,5-triazine;
2-Iodo-8-(2-methoxybenzyl)-4-(N-methylamino)pyrazolo[1,5-a]-1,3,5-triazine;
2-Bromo-8-(2-methoxybenzyl)-4-(N-methylamino)pyrazolo[1,5-a]-1,3,5-triazine;
8-[(Hydroxy)(2-thienyl)methyl]-4-(N-methyl-N-phenylamino)-2-n-propylpyrazolo[1,5-a]-1,3,5-triazine;
8-(2-Chlorobenzoyl)-2-trifluoromethyl-4-(N-methylamino)pyrazolo[1,5-a]-1,3,5-triazine;
8-(2-Chlorobenzoyl)-2-pentafluoroethyl-4-(N-methylamino)pyrazolo[1,5-a]-1,3,5-triazine;
8-(2-Chlorobenzoyl)-2-trifluoromethyl-4-(N-cyclopropylamino)pyrazolo[1,5-a]-1,3,5-triazine;
4-(N-Methyl-N-phenylamino)-2-n-propyl-8-(2-thienylmethyl)pyrazolo[1,5-a]-1,3,5-triazine;
4-(N-Methylamino)-2-n-propyl-8-[(2-thienyl)methyl]-pyrazolo[1,5-a]-1,3,5-triazine;
4-(N-Methylamino)-2-trifluoromethyl-8-[(2-thienyl)methyl]pyrazolo[1,5-a]-1,3,5-triazine;
4-(N-Cyclopropylamino)-2-trifluoromethyl-8-[(2-thienyl)methyl]pyrazolo[1,5-a]-1,3,5-triazine;
N-[2-(3,4-Dihydroxyphenyl)ethyl]-3-[4-(N-methyl-N-phenylamino)pyrazolo[1,5-a]-1,3,5-triazin-8-yl]propionamide;
3-[4-(N-Methyl-N-phenylamino)pyrazolo[1,5-a]-1,3,5-triazin-8-yl]-N-[3-(2-oxopyrrolidin-1-yl)propyl]propionamide;
N-[2-Hydroxy-2-(3,4-dihydroxyphenyl)ethyl]-3-[4-(N-methyl-N-phenylamino)pyrazolo[1,5-a]-1,3,5-triazin-8-yl]propionamide;
3-(4-Oxopyrazolo[1,5-a]-1,3,5-triazin-8-yl)propionic acid;
Ethyl 3-[4-oxopyrazolo[1,5-a]-1,3,5-triazin-8-yl]acrylate;
Sodium 4-[(hydroxy)[4-oxopyrazolo[1,5-a]-1,3,5-triazin-8-yl]methyl]benzoate;
Sodium 4-[[1-(oxo)-4-3-(oxopyrazolo[1,5-a]-1,3,5-triazin-8-yl)propyl]amino]benzoate;
Sodium 4-[2-(4-oxopyrazolo[1,5-a]-1,3,5-triazin-8-yl)-ethylsulfonylamino]benzoate;
Sodium 4-[1-oxo-3-(2-amino-4-oxopyrazolo[1,5-a]-1,3,5-triazin-8-yl)propylamino]benzoate;
Sodium 4-[1-oxo-3-(2-n-propyl-4-oxopyrazolo[1,5-a]-1,3,5-triazin-8-yl)propylamino]benzoate;
Sodium 4-[1-oxo-3-(2-trifluoromethyl-4-oxopyrazolo[1,5-a]-1,3,5-triazin-8-yl)propylamino]benzoate;
N-[2-(Indol-3-yl)ethyl]-3-(4-oxopyrazolo[1,5-a]-1,3,5-triazin-8-yl)propanamide;
N-[2-(Indol-3-yl)ethyl]-3-(2-amino-4-oxopyrazolo[1,5-a]-1,3,5-triazin-8-yl)propanamide;
N-[1-(Carboxyl)-2-(indol-3-yl)ethyl]-3-(4-oxopyrazolo-[1,5-a]-1,3,5-triazin-8-yl)propanamide;
N-[2-(4-Hydroxyphenyl)ethyl]-3-(4-oxopyrazolo[1,5-a]-1,3,5-triazin-8-yl)propanamide;
N-[2-(4-Hydroxyphenyl)ethyl]-3-(2-amino-4-oxopyrazolo-[1,5-a]-1,3,5-triazin-8-yl)propanamide;
N-[2-(4-Hydroxyphenyl)ethyl]-3-(2-trifluoromethyl-4-oxopyrazolo[1,5-a]-1,3,5-triazin-8-yl)propanamide;
N-[1-(Carboxyl)-2-(4-hydroxyphenyl)ethyl]-3-(4-oxopyrazolo-[1,5-a]-1,3,5-triazin-8-yl)propanamide;
4-(N-Methyl-N-phenylamino)pyrazolo[1,5-a]-1,3,5-triazine.
2-(4-Methylbenzyl)-8-(2-oxohept-3-yl)pyrazolo[1,5-a]-1,3,5-triazin-4-one;
8-(2-Hydroxy-6-phenylhex-3-yl)-2-(3,4-dimethoxybenzyl)pyrazolo[1,5-a]-1,3,5-triazin-4-one;
Erythro-8-(2-hydroxy-3-nonyl)pyrazolo[1,5-a]-1,3,5-triazin-4-one;
Erythro-4-amino-8-(2-hydroxy-3-nonyl)pyrazolo[1,5-a]-1,3,5-triazine;
Sodium 4-[[3-(1-methyl-4-oxopyrazolo[1,5-a]-1,3,5-triazin-8-yl)-1-(oxo)propyl]amino]benzoate;
8-Benzoyl-2-cyclopropylpyrazolo[1,5-a]-1,3,5-triazin-4-one;
N-[2-(3,4-Dihydroxyphenyl)ethyl]-3-(4-oxopyrazolo[1,5-a]-1,3,5-triazin-8-yl)propionamide;
3-[4-Oxopyrazolo[1,5-a]-1,3,5-triazin-8-yl)-N-[3-(2-oxo-pyrrolidin-1-yl)propyl]propionamide;
N-[2-Hydroxy-2-(3,4-dihydroxyphenyl)ethyl]-3-[4-oxo-pyrazolo[1,5-a]-1,3,5-triazin-8-yl]propionamide;
8-(2′-Deoxy-β-D-ribofuranosyl)-4-(N-methyl-N-phenylamino)pyrazolo[1,5-a]-1,3,5-triazine;
8-(2′-Deoxy-β-D-ribofuranosyl)-4-[N-methyl-N-(4-nitrophenylamino)]pyrazolo[1,5-a]-1,3,5-triazine;
8-(2′-Deoxy-β-D-xylofuranosyl)-4-(N-methyl-N-phenylamino)pyrazolo[1,5-a]-1,3,5-triazine;
8-(2′-Deoxy-β-D-xylofuranosyl)-4-[N-methyl-N-(4-nitro-phenylamino)]pyrazolo[1,5-a]-1,3,5-triazine;
4-Amino-8-(2′-deoxy-β-D-ribofuranosyl)pyrazolo[1,5-a]-1,3,5-triazine;
8-(2′-Deoxy-β-D-ribofuranosyl)pyrazolo[1,5-a]-1,3,5-triazin-4-one;
4-Amino-8-(2′-deoxy-β-D-xylofuranosyl)pyrazolo[1,5-a]-1,3,5-triazine;
8-(2′-Deoxy-β-D-xylofuranosyl)pyrazolo[1,5-a]-1,3,5-triazin-4-one;
4-Amino-2-fluoro-8-[trans-2,trans-3-dihydroxy-4-(hydroxymethyl)cyclopent-4-enyl]pyrazolo[1,5-a]-1,3,5-triazine;
4-Amino-8-[trans-2,trans-3-dihydroxy-4-(hydroxymethyl)-cyclopent-4-enyl]pyrazolo[1,5-a]-1,3,5-triazine;
2-Fluoro-8-[trans-2,trans-3-dihydroxy-4-(hydroxymethyl)cyclopent-4-enyl]pyrazolo[1,5-a]-1,3,5-triazin-4-one;
8-[trans-2,trans-3-dihydroxy-4-(hydroxymethyl)cyclopent-4-enyl]pyrazolo[1,5-a]-1,3,5-triazin-4-one;
(1S,4R)-2-Amino-4-(cyclopropylamino)-8-[4-(hydroxymethyl)cyclopent-2-en-1-yl]pyrazolo[1,5-a]-1,3,5-triazine;
cis-2-Amino-4-(cyclopropylamino)-8-[4-(hydroxymethyl)cyclopent-2-en-1-yl]pyrazolo[1,5-a]-1,3,5-triazine;
4-Amino-7-chloro-8-(β-D-ribofuranosyl)pyrazolo[1,5-a]-1,3,5-triazine-3′,5′-cyclophosphate;
bis-(2,2,2-Trifluoroethyl [2-[2-amino-4-(4-methoxyphenylthio)pyrazolo[1,5-a]-1,3,5-triazin-8-yl]ethoxy]-methylphosphonate;
4-Amino-8-(3′-deoxy-β-D-ribofuranosyl)pyrazolo[1,5-a]-1,3,5-triazine;
8-(3′-Deoxy-β-D-ribofuranosyl)pyrazolo[1,5-a]-1,3,5-triazin-4-one;
2-Amino-8-(3′-deoxy-β-D-ribofuranosyl)pyrazolo[1,5-a]-1,3,5-triazin-4-one;
4-Amino-2-chloro-8-(2′-deoxy-β-D-ribofuranosyl)-pyrazolo[1,5-a]-1,3,5-triazine;
cis-2-Amino-4-(cyclopropylamino)-8-[2-(hydroxymethyl)-1,3-dioxolan-4-yl]pyrazolo[1,5-a]-1,3,5-triazine;
4-Amino-8-(2′,3′-dideoxy-2′-fluoro-β-D-ribofuranosyl)-pyrazolo[1,5-a]-1,3,5-triazine;
4-Amino-8-(2′,3′-dideoxy-2′-fluoroarabinosyl)pyrazolo[1,5-a]-1,3,5-triazine;
2-Amino-8-[4-acetyloxy-3-(acetyloxymethyl)butyl]-pyrazolo[1,5-a]-1,3,5-triazine;
4-Amino-2-chloro-8-(2′-deoxy-2′-fluoro-β-D-ribofuranosyl)pyrazolo[1,5-a]-1,3,5-triazine;
4-Amino-8-(2′-deoxy-2′-fluoro-β-D-ribofuranosyl)-pyrazolo[1,5-a]-1,3,5-triazine;
8-(2′-Deoxy-2′-fluoro-β-D-ribofuranosyl)pyrazolo[1,5-a]-1,3,5-triazin-4-one;
S-[[4-Amino-8-(5′-deoxy-β-D-ribofuranosyl)pyrazolo[1,5-a]-1,3,5-triazine]-5′-yl]methionine (bioisostere of S-adenosylmethionine);
2-Amino-4-[(4-bromo-2-thienyl)methoxy]pyrazolo[1,5-a]-1,3,5-triazine;
(R)-4-Benzylamino-2-[1-(hydroxymethyl)propylamino]-8-isopropylpyrazolo[1,5-a]-1,3,5-triazine;
(S)-4-Benzylamino-2-[1-(hydroxymethyl)propylamino]-8-isopropylpyrazolo[1,5-a]-1,3,5-triazine;
2′-(Butyryl)-4-(N-butyrylamino)-8-(β-D-ribofuranosyl)-pyrazolo[1,5-a]-1,3,5-triazine-3′,5′-cyclophosphate;
cis-2,4-Diamino-8-[2-(hydroxymethyl)-1,3-dioxolan-4-yl]pyrazolo[1,5-a]-1,3,5-triazine;
cis-2-Amino-8-[2-(hydroxymethyl)-1,3-dioxolan-4-yl]-pyrazolo[1,5-a]-1,3,5-triazin-4-one;
cis-8-[2-(Hydroxymethyl)-1,3-dioxolan-4-yl]pyrazolo[1,5-a]-1,3,5-triazin-4-one;
cis-4-Amino-8-[2-(hydroxymethyl)-1,3-dioxolan-4-yl]-pyrazolo[1,5-a]-1,3,5-triazine;
(1′S,2′R)-2-Amino-8-[[1′,2′-bis(hydroxymethyl)cycloprop-1′-yl]methyl]pyrazolo[1,5-a]-1,3,5-triazin-4-one;
(1′S,2′R)-8-[[1′,2′-bis(Hydroxymethyl)cycloprop-1′-yl]-methyl]pyrazolo[1,5-a]-1,3,5-triazin-4-one;
(1′S,2′R)-4-Amino-8-[[1′,2′-bis(hydroxymethyl)cycloprop-1′-yl]methyl]pyrazolo[1,5-a]-1,3,5-triazine;
2-Amino-8-[(2-hydroxyethoxy)methyl]pyrazolo[1,5-a]-1,3,5-triazin-4-one;
8-[(2-Hydroxyethoxy)methyl]pyrazolo[1,5-a]-1,3,5-triazin-4-one;
4-Amino-8-[(2-hydroxyethoxy)methyl]pyrazolo[1,5-a]-1,3,5-triazine;
2-Amino-8-[4-hydroxy-3-(hydroxymethyl)butyl]pyrazolo[1,5-a]-1,3,5-triazin-4-one;
4-Amino-8-[4-hydroxy-3-(hydroxymethyl)butyl]pyrazolo[1,5-a]-1,3,5-triazine;
8-[4-Hydroxy-3-(hydroxymethyl)butyl]pyrazolo[1,5-a]-1,3,5-triazin-4-one;
2-Amino-8-[2-hydroxy-1-(hydroxymethyl)ethoxymethyl]-pyrazolo[1,5-a]-1,3,5-triazin-4-one;
8-[2-Hydroxy-1-(hydroxymethyl)ethoxymethyl]pyrazolo[1,5-a]-1,3,5-triazin-4-one;
4-Amino-8-[2-hydroxy-1-(hydroxymethyl)ethoxymethyl]-pyrazolo[1,5-a]-1,3,5-triazine;
2-[(2-Amino-4-oxopyrazolo[1,5-a]-1,3,5-triazin-8-yl)methoxy]ethyl valinate;
8-(2′,3′-Dideoxy-β-D-ribofuranosyl)pyrazolo[1,5-a]-1,3,5-triazin-4-one;
8-(2′,3′-Dideoxy-2′,2′-difluoro-β-D-ribofuranosyl)-pyrazolo[1,5-a]-1,3,5-triazin-4-one;
8-(2′-Deoxy-β-D-ribofuranosyl)pyrazolo[1,5-a]-1,3,5-triazin-4-one;
bis(Pivaloyloxymethyl) [2-(4-aminopyrazolo[1,5-a]-1,3,5-triazin-8-yl)ethoxy]methylphosphonate;
Sodium [2-(4-aminopyrazolo[1,5-a]-1,3,5-triazin-8-yl)-ethoxy]methylphosphonate;
4-Amino-8-[2-[[bis(pivaloyloxymethyl)phosphonyl]-methoxy]ethyl]pyrazolo[1,5-a]-1,3,5-triazine;
cis-8-[2-(Hydroxymethyl)-1,3-oxathiolan-5-yl]pyrazolo[1,5-a]-1,3,5-triazin-4-one;
cis-8-[2-(Hydroxymethyl)-1,3-oxathiolan-5-yl]-2-oxo-pyrazolo[1,5-a]-1,3,5-triazin-4-one;
cis-8-[2-(Hydroxymethyl)-1,3-oxathiolan-5-yl]-2-thioxopyrazolo[1,5-a]-1,3,5-triazin-4-one;
cis-2-Amino-8-[2-(hydroxymethyl)-1,3-oxathiolan-5-yl]-pyrazolo[1,5-a]-1,3,5-triazin-4-one;
cis-4-Amino-8-[2-(hydroxymethyl)-1,3-oxathiolan-5-yl]-pyrazolo[1,5-a]-1,3,5-triazine;
8-[[3R,4R)-3-Hydroxy-4-(hydroxymethyl)pyrrolidin-1-yl]-methyl]pyrazolo[1,5-a]-1,3,5-triazin-4-one; and
4-Amino-8-[[(3R,4R)-3-hydroxy-4-(hydroxymethyl)pyrrolidin-1-yl]methyl]pyrazolo[1,5-a]-1,3,5-triazine.

10. A pharmaceutical composition comprising the compound of claims 1 or 9, wherein said compound is combined with a pharmaceutically acceptable vehicle or excipient.

11. A method for preparing the compound of claim 1 in which R1=H, comprising:
a) reacting a compound of general formula (V) 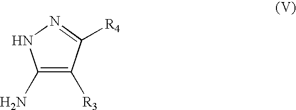
in which R3 is as defined in claim 3 and R4 is H, with a compound of formula R2C(GP)═NH, in which R2 is as defined in claim 3 and GP represents a leaving group, so as to obtain a compound of formula (VI) 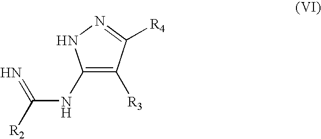
and
b) reacting the compound of formula (VI) with a dielectrophile of ethyl carbonate type or of orthoester type so as to obtain a compound of formula (Ia) or (Ib).

12. The method of claim 11 wherein in step a) the compound of formula (V) is reacted with an imidate of formula R2(OMe)=NH.HCl and in step b), the compound obtained in a) is reacted with an ethyl carbonate so as to obtain a compound of formula (VII) 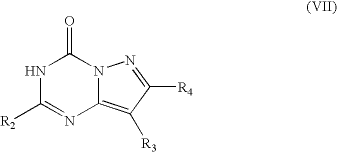
which can optionally be reacted with phosphorus oxychloride and a tertiary amine so as to obtain a compound of formula (VIII) 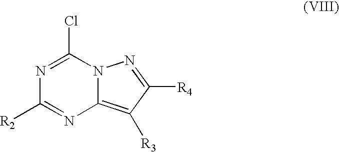
which can optionally be reacted with an amine of formula HNRxRy so as to obtain a compound of formula (Ib) in which Y═NRxRy, where Y, Rx and Ry are as defined in claim 1.

13. The method of claim 12 wherein Y represents an N-methyl-N-phenylamino group, and the compound (Ib) is treated with a hydroxide so as to obtain a compound of formula (Ib) in which Y═OH.

14. A medicinal product intended to increase the secretion of one or more neurotrophic factors for treating or preventing pathologies involving neuronal degeneration comprising:
8-(1-hydroxypropyl)-2-methyl-4-(N-methyl-N-phenylamino)pyrazolo[1,5-a]-1,3,5-triazine, ethyl 2-methyl-4-(N-methyl-N-phenylamino)pyrazolo[1,5-a]-1,3,5-triazine-6-carboxylate, 2-methyl-4-(N-methyl-N-phenylamino)-8-phenylpyrazolo[1,5-a]-1,3,5-triazine, 2-methyl-4-(N-methylamino)-8-(prop-1-ynyl)pyrazolo[1,5-a]-1,3,5-triazine, 2-methyl-4-(N-methyl-N-phenylamino)-8-(β-D-glycero-pentofuran-3′-ulos-1′-yl)pyrazolo[1,5-a]-1,3,5-triazine, 2-methyl-4-(methylamino)pyrazolo[1,5-a]-1,3,5-triazine, 2-methyl-4-[4-(N,N-dimethylaminophenyl)]pyrazolo[1,5-a]-1,3,5-triazine, pyrazolo-[1,5-a]-1,3,5-triazin-4-one, 2-methylpyrazolo[1,5-a]-1,3,5-triazin-4-one, 2-thioxo-1,2,3,4-tetrahydropyrazolo[1,5-a]-1,3,5-triazin-4-one, 2-thiomethylpyrazolo[1,5-a]-1,3,5-triazin-4-one, 2-methyl-4-(N-methyl-N-phenylamino)pyrazolo[1,5-a]-1,3,5-triazine, 2-methyl-4-[N-methyl-N-(4-nitrophenyl)amino]-8-nitropyrazolo[1,5-a]-1,3,5-triazine, 8-amino-4-[N-(4-aminophenyl)-N-methylamino]-2-methylpyrazolo[1,5-a]-1,3,5-triazine, 8-acetamido-4-[N-(4-acetamidophenyl)-N-methylamino]-2-methylpyrazolo[1,5-a]-1,3,5-triazine, 8-iodo-2-methyl-4-(N-methyl-N-phenylamino)pyrazolo[1,5-a]-1,3,5-triazine, 8-[(hydroxy)(phenyl)methyl]-2-methyl-4-(N-methyl-N-phenylamino)pyrazolo[1,5-a]-1,3,5-triazine, 8-benzyl-2-methyl-4-(N-methyl-N-phenylamino)pyrazolo[1,5-a]-1,3,5-triazine, 8-benzoyl-2-methyl-4-(N-methyl-N-phenylamino)pyrazolo[1,5-a]-1,3,5-triazine, N,N-diethyl-2-methyl-4-(N-methyl-N-phenylamino)-pyrazolo[1,5-a]-1,3,5-triazine-6-carboxamide, 8-benzyl-2-methylpyrazolo[1,5-a]-1,3,5-triazin-4-one and 8-benzoyl-2-methylpyrazolo[1,5-a]-1,3,5-triazin-4-one.

15. A method for increasing the secretion of one or more neurotrophic factors for treating or preventing pathologies involving neuronal degeneration comprising administration of the compound selected from:
8-(1-hydroxypropyl)-2-methyl-4-(N-methyl-N-phenylamino)pyrazolo[1,5-a]-1,3,5-triazine, ethyl 2-methyl-4-(N-methyl-N-phenylamino)pyrazolo[1,5-a]-1,3,5-triazine-8-carboxylate, 2-methyl-4-(N-methyl-N-phenylamino)-8-phenylpyrazolo[1,5-a]-1,3,5-triazine, 2-methyl-4-(N-methylamino)-8-(prop-1-ynyl)pyrazolo[1,5-a]-1,3,5-triazine, 2-methyl-4-(N-methyl-N-phenylamino)-8-(β-D-glycero-pentofuran-3′-ulos-1′-yl)pyrazolo[1,5-a]-1,3,5-triazine, 2-methyl-4-(methylamino)pyrazolo[1,5-a]-1,3,5-triazine, 2-methyl-4-[4-(N,N-dimethylaminophenyl)]pyrazolo[1,5-a]-1,3,5-triazine, pyrazolo-[1,5-a]-1,3,5-triazin-4-one, 2-methylpyrazolo[1,5-a]-1,3,5-triazin-4-one, 2-thioxo-1,2,3,4-tetrahydropyrazolo[1,5-a]-1,3,5-triazin-4-one, 2-thiomethylpyrazolo[1,5-a]-1,3,5-triazin-4-one, 2-thiomethyl-4-(N-methyl-N-phenylamino)pyrazolo[1,5-a]-1,3,5-triazine, 2-methyl-4-[N-methyl-N-(4-nitrophenyl)amino]-8-nitropyrazolo[1,5-a]-1,3,5-triazine, 8-amino-4-[N-(4-aminophenyl)-N-methylamino]-2-methylpyrazolo[1,5-a]-1,3,5-triazine, 8-acetamido-4-[N-(4-acetamidophenyl)-N-methylamino]-2-methylpyrazolo[1,5-a]-1,3,5-triazine, 8-iodo-2-thiomethyl-4-(N-methyl-N-phenylamino)pyrazolo[1,5-a]-1,3,5-triazine, 8-[(hydroxy)(phenyl)methyl]-2-methyl-4-(N-methyl-N-phenylamino)pyrazolo[1,5-a]-1,3,5-triazine, 8-benzyl-2-methyl-4-(N-methyl-N-phenylamino)pyrazolo[1,5-a]-1,3,5-triazine, 8-benzoyl-2-methyl-4-(N-methyl-N-phenylamino)pyrazolo[1,5-a]-1,3,5-triazine, N,N-diethyl-2-methyl-4-(N-methyl-N-phenylamino)-pyrazolo[1,5-a]-1,3,5-triazine-8-carboxamide, 8-benzyl-2-methylpyrazolo[1,5-a]-1,3,5-triazin-4-one and 8-benzoyl-2-methylpyrazolo[1,5-a]-1,3,5-triazin-4-one.

16. A pharmaceutical composition containing a compound according to claim 4 wherein said compound has a structure represented by formulae (Ic1) and (Ic2) 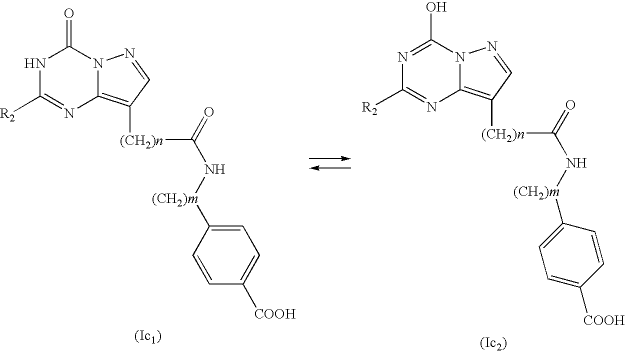
or its pharmaceutically acceptable base or acid addition salts, wherein: R2 represents a hydrogen atom, n=2 and m=0.

17. A method of treating or preventing pathologies involving neuronal degeneration comprising:
administration of the compound of claims 1 or 9, including compounds corresponding to the following formulae: 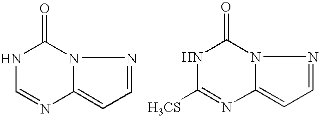
wherein said pathology involving neuronal degeneration is aging, senility, Alzheimer's disease, Parkinson's disease, amyotrophic lateral sclerosis, multiple scleroses, Huntington's disease, Down's syndrome, cerebral strokes, peripheral neuropathies, retinopathies (in particular pigmentary retinitis), prion diseases (in particular spongiform encephalopathies of the Creutzfeldt-Jakob disease type), traumas (accidents to the vertebral column, compression of the optic nerve subsequent to a glaucoma, etc.), or a neuronal disorder caused by the action of chemical products and nerve lesions.

18. A method for treating or preventing central or peripheral diseases in a mammal comprising administration to said mammal of a compound comprising:
administration of the compound of claims 1 or 9 to said mammal, including compounds corresponding to the following formulae:

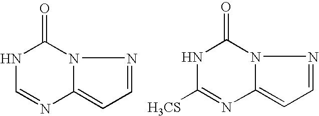

19. A medicinal product for inhibiting a phosphodiesterase type 2 or 4 comprising the compound of claims 1 or 9, including compounds corresponding to the following formulae:

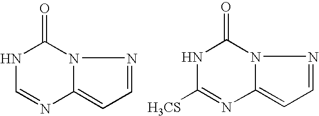

20. A method of treating a mammal comprising: administration of the medicinal product of claim 19 to said mammal, wherein said medicinal product is an antimicrobial, antiviral or anticancer medicinal product, or a medicinal product having cardiovascular effects.

21. A method of treating or preventing central or peripheral diseases in a mammal comprising:
administration of an effective amount of a pharmaceutical composition including the compound of claims 1 or 9, including compounds corresponding to the following formulae: 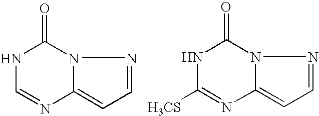
wherein said central or peripheral disease is an inflammatory disease, chronic obstructive bronchopathies, rhinitis, dementia, acute respiratory distress syndrome, allergies, dermatitis, psoriasis, rheumatoid arthritis, infections, viral infections, autoimmune diseases, multiple scleroses, in particular multiple sclerosis, dyskinesias, glomerulonephritis, osteoarthritis, cancer, septic shock, AIDS, Crohn's disease, osteoporosis, rheumatoid arthritis, obesity, depression, anxiety, schizophrenia, bipolar disorder, attention deficits, fibromyalgia, Parkinson's disease and Alzheimer's disease, diabetes, amyotrophic sclerosis, multiple scleroses, Lewy body dementias, conditions with spasms such as epilepsy, fibromyalgia, central nervous system pathologies associated with senescence, memory disorders, or psychiatric disorder.
